(19) United States
(12) Patent Application Publication
(10) Pub. No.: US 2024/0316878 A1
(43) Pub. Date: Sep. 26, 2024

(54) MANUFACTURE OF MICROSTRUCTURES

(71) Applicant: TransDerm, Inc., North Chicago, IL (US)

(72) Inventors: Tycho J. Speaker, Santa Cruz, CA (US); Manuel A. Flores, Santa Cruz, CA (US); Novejot K. Singh, Hayward, CA (US); Abigail L. Lambretti, Santa Cruz, CA (US); Anna C. Henderson, Santa Cruz, CA (US); Tyler A. Stewart, Scotts Valley, CA (US); Andrea S. Burgon, Ben Lomond, CA (US)

(73) Assignee: TransDerm, Inc., North Chicago, IL (US)

(21) Appl. No.: 18/517,351

(22) Filed: Nov. 22, 2023

Related U.S. Application Data
(63) Continuation of application No. 17/111,439, filed on Dec. 3, 2020, now abandoned.
(60) Provisional application No. 62/943,152, filed on Dec. 3, 2019.
(60) Provisional application No. 62/943,153, filed on Dec. 3, 2019.

(51) Int. Cl. B29C 67/00; A61M 37/00; B29C 69/00; B29K 29/00; B29L 31/00; C08J 5/18; C08L 5/00; C08L 29/04

(52) U.S. Cl. CPC B29C 67/0011; A61M 37/0015; B29C 69/001; C08J 5/18; C08L 5/00; C08L 29/04; A61M 2037/0023; A61M 2037/0053; A61M 2207/10; B29K 2029/04; B29L 2031/7544; C08J 2305/00; C08J 2329/04

(57) ABSTRACT

Microstructure manufacturing apparatuses and methods are disclosed herein that enable the production of microstructures in an efficient, cost-effective manner that produces precise, high-quality microstructure products. In the manufacturing process for a microneedle array, for example, a template can be contacted against a surface of a continuous layer of a viscous polymer and separated from the surface to form a plurality of projections. The projections can then be solidified and laser cut at a predetermined distance from the surface to form the microstructure.

### CROSS-REFERENCE TO RELATED APPLICATIONS

This application is a continuation of U.S. patent application Ser. No. 17/111,439 filed Dec. 3, 2020 and claims the benefit of U.S. Provisional Application No. 62/943,152, filed Dec. 3, 2019, and U.S. Provisional Application No. 62/943,153, filed Dec. 3, 2019, the entireties of each of which is incorporated herein by reference.

### BACKGROUND

This disclosure relates generally to the field of drug delivery devices, and in particular, to devices used for transdermal delivery of therapeutic agents and methods and apparatuses for manufacturing the same.

Microneedles and microneedle patches have been contemplated as mechanisms for transdermal delivery of various therapeutic agents. For effective and efficient delivery of therapeutic agents, microneedles need to have the right combination of materials, size, and mechanical properties. However, manufacturing and, in particular, mass manufacturing of microneedles with the appropriate combination of these factors remains a challenge.

### SUMMARY

In accordance with some embodiments disclosed herein is the realization that it is difficult to manufacture a microstructure, such as a microneedle array, that is suitable for transdermal delivery of therapeutic agents at a commercially viable throughput and yield. For example, it has been discovered that there are substantial manufacturing challenges due to the size and aspect ratio of individual microstructures, such as microneedles within an array, especially when trying to achieve advantageous physical properties and specific microstructure arrangements, as discussed further herein. In order to address this and other challenges, the apparatuses and methods disclosed herein enable high-throughput mass manufacturing of microstructures, such as microneedle arrays, while avoiding breakage and waste and while facilitating advantageous microstructure arrangements and geometries using a variety of material and/or active agent combinations.

For example, some embodiments provide for a method of manufacturing a microneedle array that may include a pulling fibers from a continuous film of a viscous polymer using a pin template while simultaneously drying the pulled fibers. The inventors of the present disclosure have discovered that in some embodiments, simultaneous drying can provide uniformity of size, viscosity and hardness of the fibers. The pulled, partially dried fibers are then cut to at a suitable length to form the microneedle array. Advantageously, some embodiments of the method can significantly minimize breakage and waste by cutting the fibers at a distal end thereof rather than removing and separating the microneedles from a mold, as done in a typical microneedle manufacturing process.

Therefore, in accordance with some embodiments, the microstructures disclosed herein can be made in an efficient, precise, assembly-line-like execution of steps. This precision manufacturing and enable production that significantly reduces risk of damage to the microstructures, substantially improves consistency and quality, thereby enabling a manufacturer employing the highest of quality controls to achieve production of a high-quality product with minimal waste product and associated expense. These and other aspects of the methods and apparatuses disclosed herein can be especially valuable considering the relatively high cost of active agents and other pharmaceuticals that may be incorporated into the microstructures.

Accordingly, in some embodiments, a microstructure can be provided that includes one or more protrusions (e.g., microneedles) extending from a sheet of solidified viscous polymer.

In some embodiments, the polymer composition can include a plasticizer. For example, the plasticizer can make the polymer more flexible in dried form, thereby reducing the interfacial stresses that develop between the rigid substrate during the formation of the microneedles by stretching and similar deformation of the polymer. The reduction in the interfacial stress makes the microneedle protrusions and the supporting layer less prone to spontaneous out-of-plane deformation and premature or spontaneous peeling or adhesion loss that can result from such deformation, thereby increasing the yield during a manufacturing process.

In accordance with some embodiments, a method of manufacturing a microstructure includes disposing a viscous polymer onto a substrate to form a continuous, viscous film layer and contacting a template having a plurality of contact points against a surface of the viscous film layer. The template is then separated from the viscous film layer while urging air towards the viscous film layer from the template to form a plurality of fiber-like projections of the viscous polymer. The projections can then be permitted to solidify and cut to form microneedles.

The air flow during the separation of the template from the viscous film layer enables a uniform drying and/or solidification of the viscous polymer, thereby increasing the uniformity of the microneedles being formed. Additionally, the air flow also increases the rate of drying, thereby increasing the throughput of the manufacturing process.

In accordance with some embodiments, an apparatus for manufacturing a microstructure includes a substrate carrier and a template holder. The substrate carrier can be configured to carry a substrate and the template holder can be configured to carry a template having a plurality of contact points and enable a flow of air through outlet apertures provided in the template. An assembly can be configured to move the substrate holder relative to the template holder so as to enable the plurality of contact points to contact a surface of a viscous polymer layer disposed on the substrate and draw the viscous polymer at points of contact between the surface of the viscous polymer and the plurality of contact points to form protrusions of the viscous polymer. The assembly is further configured to permit the protrusions to solidify in place.

The apparatus disclosed herein enables faster manufacturing of microneedles. Additionally, the apparatus improves the uniformity of the manufacturing, thereby increasing the yield and precision of manufacturing.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF DRAWINGS

In the present disclosure, reference is made to the accompanying drawings, which form a part hereof. In the drawings, similar symbols typically identify similar components, unless context dictates otherwise. The illustrative embodiments described in the detailed description, drawings, and claims are not meant to be limiting. Some embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

FIG. 1 illustrates a microstructure having one or more microneedles coupled to an underlying substrate, according to some embodiments.

FIG. 2 illustrates an apparatus used for manufacturing microstructures, in accordance with some embodiments.

FIG. 3 illustrates a portion of the apparatus for manufacturing microstructures, in accordance with some embodiments.

FIG. 4 illustrates a template mount and a substrate holder, in accordance with some embodiments.

FIG. 5 illustrates a template mount and a substrate holder accommodating an overdrive of the substrate holder, in accordance with some embodiments.

FIG. 6 shows a perspective view of an airflow ingress channel, in accordance with some embodiments.

FIG. 7 shows a perspective bottom view of an airflow ingress channel in conjunction with a template, in accordance with some embodiments.

FIG. 8 shows a perspective view of a template used for manufacturing microstructures, in accordance with some embodiments.

FIG. 9 shows a top view of a template used for manufacturing microstructures, in accordance with some embodiments.

FIG. 10 shows a side view of a template used for manufacturing microstructures, in accordance with some embodiments.

FIG. 11 shows an enlarged perspective view of a template used for manufacturing microstructures, in accordance with some embodiments.

FIG. 12 shows an enlarged bottom view of a template used for manufacturing microstructures, in accordance with some embodiments.

FIGS. 13A and 13B illustrate a method of forming a continuous layer of a viscous polymer with uniform thickness, in accordance with some embodiments.

FIGS. 14-22 illustrate the apparatus for manufacturing the microstructure during various stages of performing a method of manufacturing the microstructure, in accordance with some embodiments.

FIG. 23 illustrates a non-contact method of loading microneedles with an active agent, in accordance with some embodiments.

FIG. 24 shows a photomicrograph of the cross-section of a microneedle loaded with a hydroalcoholic solution of sodium fluorescein, in accordance with some embodiments.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding.

Some embodiments of the present disclosure relate to microstructures for transdermal delivery of therapeutic agents, methods of manufacturing such microstructures, apparatuses for manufacturing such microstructures, and methods of treatment using such microstructures. The microstructures can be useful in a variety of treatments and indications. Such microstructures can be incorporated into or used in combination with other treatment modalities and medical devices.

Among the various advantages and benefits disclosed herein, in some embodiments, the manufacturing apparatus can reliably produce microstructures at a commercially viable throughput and yield. The methods and apparatuses disclosed herein can produce microstructures, such as microneedle arrays, in a manner that is more cost efficient and effective than prior methods and apparatuses. As a result, various advantages and benefits, including certain features of the microstructure, as disclosed herein, can be achieved and realized whereas such advantages and benefits were formerly not possible.

In accordance with some embodiments, the manufacturing apparatus can comprise an airflow mechanism that directs air during the manufacturing process toward the microstructures to enable a uniform drying and/or solidification of the viscous polymer forming the microstructures. Further, various methods, systems, and microstructures are disclosed herein that facilitate precise manufacturing, drug loading, microstructure removal, separation, and packaging, which can each make possible various advantageous, cost-saving, and quality-improving benefits, as noted here.

### Microstructures

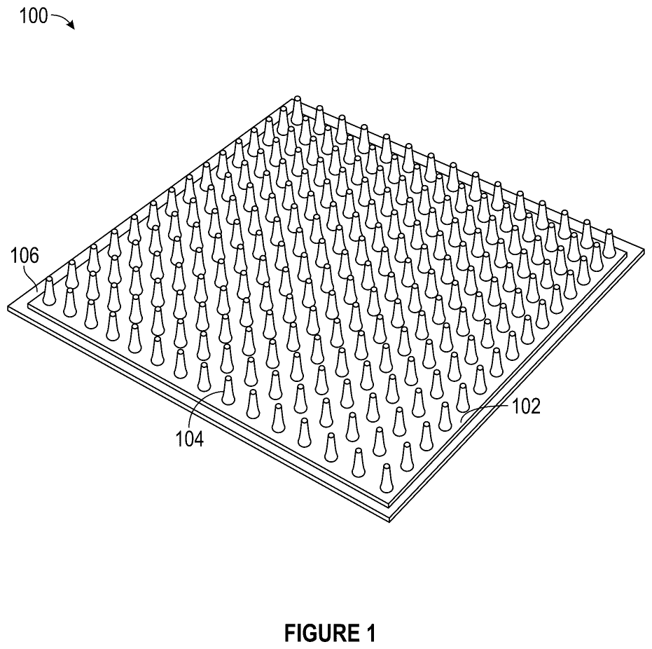

Referring now to the figures, FIG. 1 illustrates a microstructure having one or more microneedles coupled to an underlying substrate. FIG. 1 shows a microneedle patch 100 having a base layer 102 and a plurality of microneedles 104 extending from the base layer 102. In some embodiments, the material for the base layer 102 can be the same as the material for the microneedles 104. Optionally, the base layer 102 and the microneedles 104 can be compositionally homogenous, i.e., formed as a monolithic, continuous piece. Some features of the microstructures disclosed herein can be implemented using aspects of the disclosures of U.S. Pat. Nos. 8,366,677 and 10,377,062, the entireties of each of which are incorporated herein by reference.

In some embodiments, the patch 100 can optionally include a substrate layer 106 disposed over the base layer 102 and the microneedles 104 are formed on the substrate layer 106. In some embodiments, the material for the substrate layer 106 and the microneedles 104 may be same. In some embodiments, the material for the microneedles 104 and the materials for the base layer 102 and/or the substrate layer 106 are different. In some embodiments, the microneedles 104 include a polymer. In some embodiments, the microneedles 104 are predominantly formed of a polymer, e.g., a water soluble polymer. The polymer can be a biocompatible or a biodegradable polymer or a polymer that undergoes physiological clearance, for example, in vertebrate animals.

In some embodiments, the microneedles 104 can comprise a therapeutic agent such as, for example, a toxin having therapeutic properties. Treatment methods using the patch 100 can be performed by applying the patch 100 to the skin of a subject such that the microneedles 104 penetrate the skin surface. In embodiments where the microneedles 104 are formed of a biodegradable polymer or a physiologically cleared polymer, the microneedles 104 may dissolve upon penetration into the skin surface. Further, in embodiments in which the microneedles 104 include or are loaded with a therapeutic agent, the therapeutic agent may be delivered to the subject upon penetration into the skin surface. In other words, the patch 100 in some embodiments can be used for transdermal delivery of a therapeutic agent to the subject.

### Manufacturing Apparatuses for Microstructures

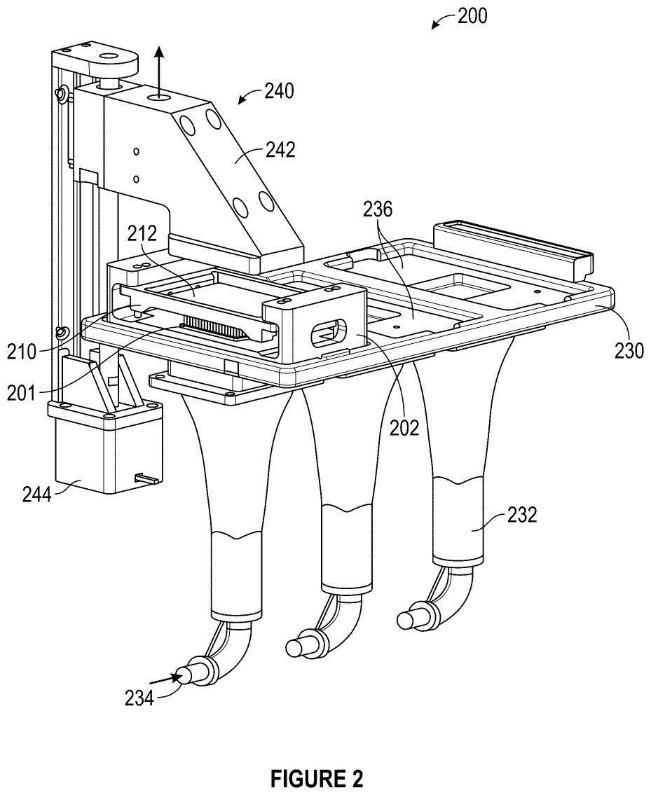

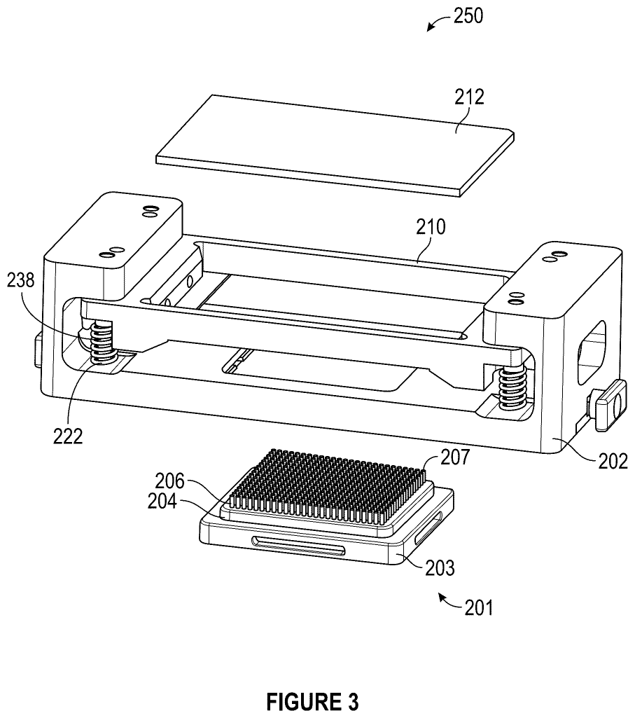

FIG. 2 and FIG. 3 illustrate a portion of an apparatus 200 used for manufacturing microstructures, in accordance with some embodiments of the present disclosure. The apparatus 200 can be used for mass manufacturing microstructures such as, for example, an array of patches 100. Moreover, the process of manufacturing the microstructures can be automated and scaled for commercial manufacturing using the apparatus 200, which can provide reliable, high-throughput manufacturing of the microstructures that are compliant with regulator standards, e.g., for therapeutic use.

Referring to FIG. 2, the apparatus 200 can include a table 230 having a plurality of slots 236 that can each be configured to hold at least one stamping assembly 250. In some embodiments, each of the slots 236 can additionally be provided with an airflow ingress channel 232. The airflow ingress channels 232 can be connected to an air hose 234 for providing forced air flow through the airflow ingress channels 232. The air flowing through the airflow ingress channels 232 may be directed to flow toward the stamping assemblies 250 during the process of manufacturing the microstructures, as will be explained in detail elsewhere herein.

An embodiment of the stamping assembly 250 is illustrated in FIG. 3, along with an embodiment of the template 201. As illustrated, the stamping assembly 250 can comprise a template mount 202 and a substrate holder 210. The template mount 202 can comprise one or more alignment arms 252 and one or more alignment pins. The alignment arms 252 and the alignment pins can support or direct movement of the substrate holder 210, tending to ensure that the substrate holder 210 remains aligned within a horizontal plane when being vertically translated during manufacture of the microstructure.

For example, the alignment arms 252 can engage with and support four separate alignment pins. The substrate holder 210 can comprise four alignment apertures, each being disposed at a respective corner of the substrate holder 210, which can receive and slide along a respective alignment pin of the stamping assembly 250. During movement of the substrate holder 210 relative to the template mount 202, the engagement between the alignment apertures of the substrate holder 210 and the alignment pins can maintain the substrate holder 210 in a horizontal plane. As will be appreciated by a person of skill in the art, the maintenance of the substrate holder 210 (and thereby the substrate 212) in a horizontal plane during the manufacturing process ensures that the microstructure can be repeatably and reliably produced. The structure and function of the stamping assembly and apparatus disclosed herein provide unique and superior benefits that achieve these objectives.

Additionally, and advantageously, the arrangement of template mount 202 and the stamping assembly 250 enables stabilization of the microneedle structures in a final position after the formation of the microneedles. Moreover, because the microneedle structures are formed relatively quickly in comparison to the complete drying to form the sufficiently rigid structures to be free-standing without collapsing, the template mount 202 may be moved away from the stamping assembly 250 to allow complete drying while additional microneedle formation steps are performed under the airflow conditions provided by the airflow ingress channels 232.

Referring again to FIG. 2, the apparatus 200 can comprise a drive assembly 240 that is configured to drive relative motion of the various components of the stamping assembly 250 during the manufacture of the microstructure. The drive assembly 240 can include one or more stamper arms 242 and a drive mechanism or actuator 244. The stamper arm 242 can be configured to engage with at least a portion of the stamping assembly 250 to facilitate the manufacturing process discussed herein.

For example, the stamper arm 242 can include a magnetic chuck or a vacuum chuck (not explicitly shown) for coupling to and moving the substrate holder 210 relative to the template mount 202, which can in turn, cause movement of the substrate 212. The coupling between the stamper arm 242 and the substrate holder 210 can allow the stamper arm 242 to maintain the substrate holder 210 in a horizontal plane while the drive assembly 240 moves stamper arm 242 (and the substrate holder 210 coupled thereto) vertically relative to the table 230 and the template mount 202. In this manner, the stamper arm 242 can move the substrate 212 toward or away from the template 201, allowing the operator to perform the manufacturing steps to form the microstructures, such as the patch 100 described above.

In some embodiments, during the process of manufacturing the microstructures, the drive assembly 240 as a whole or the stamper arm 242 alone may be moved laterally from one slot 236 to another slot 236 to allow engagement different stamping assemblies. This can advantageously permit the apparatus 200 to perform mass manufacturing of the microstructures. The configuration and components of the apparatus 200 can also be built to a scale suitable to support a desired manufacturing output using the principles disclosed herein.

In some embodiments, the actuator 244 can comprise one or more actuators or motors that are configured to provide both course and fine movement or adjustments to the stamper arm 242, whether in vertical and/or horizontal direction(s). For example, the actuator 244 can move the stamper arm 242 with a precision of between about 0.1 μm to about 2.5 μm over a distance in a range from about 50 μm to about 500 mm, such as from about 50 μm to about 100 μm, from about 50 μm to about 250 μm, from about 50 μm to about 500 μm, from about 50 μm to about 1000 μm, from about 50 μm to about 2 mm, from about 50 μm to about 5 mm, from about 50 μm to about 10 mm, from about 50 μm to about 50 mm, from about 50 μm to about 100 mm, from about 50 μm, to about 250 mm, from about 100 μm to about 250 μm, from about 100 μm to about 500 μm, from about 100 μm to about 1 mm, from about 100 μm to about 5 mm, from about 100 μm to about 10 mm, from about 100 μm to about 50 mm, from about 100 μm to about 100 mm, from about 500 μm to about 1 mm, from about 500 μm to about 5 mm, from about 500 μm to about 50 mm, or any other range between any two of these ranges or any distance within any of these ranges, and at rates ranging from about 0.1 mm/minute to about 200 mm/minute, e.g., from about 0.1 mm/minute to about 1 mm/minute, about 0.1 mm/minute to about 2.5 mm/minute, from about 0.1 mm/minute to about 5 mm/minute, from about 0.1 mm/minute to about 10 mm/minute, from about 0.1 mm/minute to about 50 mm/minute, from about 0.1 mm/minute to about 100 mm/minute, from about 1 mm/minute to about 10 mm/minute, from about 1 mm/minute to about 20 mm/minute, from about 1 mm/minute to about 50 mm/minute, from about 1 mm/minute to about 100 mm/minute, or any other range between any two of these ranges or any rate within any of these ranges. It will be appreciated that the optimal rate at which the substrate 212 is separated from the template 201 will depend on several factors such as, for example, the composition of the viscous polymer disposed on the substrate 212, the molecular weight of the viscous polymer, the amount of solvent present in the viscous polymer, etc. that determine the rheological characteristics of the polymer.

In some embodiments, the actuator 244 may include, for example, a pneumatic, hydraulic, magnetic, electrical, piezoelectric or other type of mechanical and/or electromechanical actuators that can move the stamper arm 242 relative to the table 230.

Referring again to FIG. 3, the stamping assembly 250 can be configured such that the template mount 202 is configured to hold a template 201, and the substrate holder 210 is configured to hold a substrate 212 on which a composition can be placed and from which the microstructures are formed. As noted above, the substrate 212 can be moved relative to the template 201 in order to form the microstructure from the composition disposed on the substrate 212.

In accordance with some embodiments, the template 201 can comprise a template holder 203, a template base 204, and an array of pins 206 mounted to and extending from the template base 204. The template 201 can be replaceably coupled to the template mount 202 of the stamping assembly 250.

In some embodiments, the template mount 202 can include a coupling device, such as magnetic or vacuum chucks, for coupling to or engaging with the template 201 to immobilize the template 201 relative to the template mount 202 during the manufacturing process. Similarly, in some embodiments, the substrate holder 210 may include a coupling device, such as magnetic or vacuum chucks, for coupling the substrate 212 to the substrate holder 210. In some embodiments, such a coupling can advantageously permit the template 201 to be fixed relative to the template mount 202 and the substrate 212 to be fixed relative to the substrate holder 210 in order to ensure that the template 201 and the substrate 212 are properly aligned.

Accordingly, some embodiments thereby permit the substrate holder 210 to be moved vertically relative to the template mount 202 in a precise and controlled manner that permits the manufacturing of a microstructure having specific structural properties. As noted above, the stamper arm 242 can engage with the substrate holder 210 for moving the substrate holder 210 vertically relative to the template 201. For example, once the template 201 and the substrate 212 are aligned with respect to each other, the stamper arm 242 may be engaged with the substrate holder 210. Thereafter, the actuator 244 can move the stamper arm 242, and thereby the substrate holder 210, toward template 201. The apparatus 200 can thereby cause contact points 207 of the pins 206 of the template 201 to come in contact with a surface of a composition, such as a viscous polymer disposed on the substrate 212.

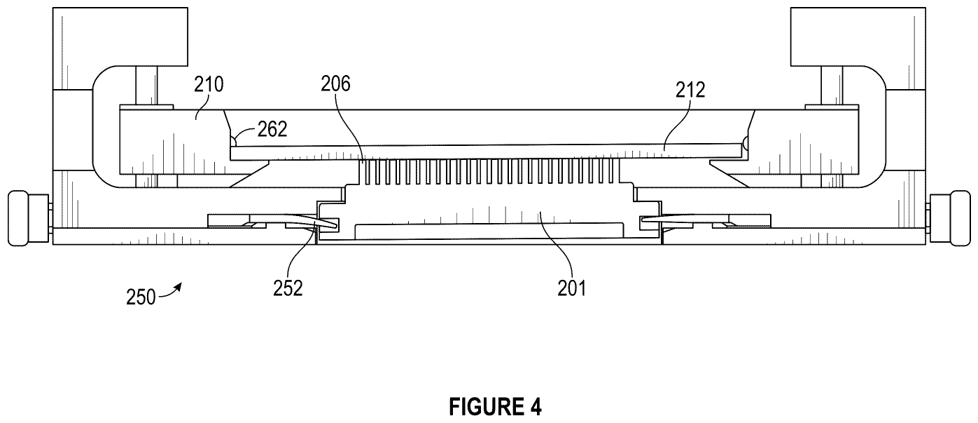

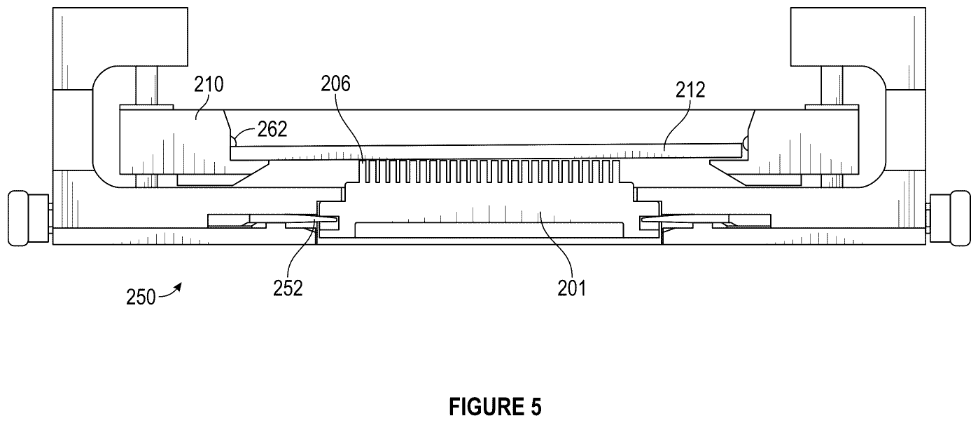

FIGS. 4 and 5 illustrate a template mount 202 in accordance with some embodiments of the present disclosure. In some embodiments, the template mount 202 may include a spring-loaded template mounting structure to hold the template 201 in place while allowing the template 201 as a whole can comply with an overdrive of the substrate 212. Compliance of the template 201 may prevent deformation of the pins 206. The template mount 202 of the stamp assembly 250 can, in some embodiments, include one or more alignment arms or spring-loaded template detent contacts 252 that capture the template 201 and allow the template 201 to move downward by a small distance if, e.g., by accident the substrate 212 is moved beyond a point at which the pins 206 of the template 201 contact the substrate 212 by an overdrive of the stamper arm 242. In some embodiments, the template detent contacts 252 can comply against an uneven force independently of each other as can be seen in FIG. 4. Such independent compliance allows the template 201 to tilt at an angle if the substrate 212 contacts the template 201 at an angle, e.g., as seen in FIG. 5. The downward and tilting movement enabled by the template detent contacts 252 prevents the pins from buckling under the force exerted by the substrate 212 (which, in some embodiments, may be rigid and non-compliant), and prevents damage to the substrate 212.

In some embodiments, the substrate holder 210 may optionally include a spring-loaded substrate mounting structure to hold the substrate 212 in place. The substrate mounting structure may include spring-loaded substrate detent contacts 260 to enable the substrate 212 to comply against the template 201 to guard against substrate overdrive. Similarly to the template detent contacts 252 discussed herein with respect to the template mount 202, the substrate detent contacts 260 allow the substrate 212 to comply and/or tilt in case of an overdrive of the substrate holder 210 by the stamper arm 242.

Advantageously, such a compliance feature of the template mount 202 and/or the substrate holder 210 affords the possibility of slightly overdriving the stamper arm 242 so as to ensure full contact of the maximum number of pin structures 206 with the substrate 212, and thereby, maximal embedding of the pin structures 206 in the polymer layer so as to produce a maximally uniform set of the resulting microstructures formed upon withdrawal of the pin template 201.

Once the composition is brought into contact with the pins 206 of the template 201, the substrate 212 can thereafter be moved away from the pins 206 of the template 201 to draw the composition into a plurality of corresponding projections or needles.

For example, in some embodiments, the pins 206 can be placed into contact with the composition disposed on the substrate 212 for a predetermined amount time. Once the predetermined amount time has elapsed, the actuator 244 can move the substrate holder 210 away from template 201 such that the viscous polymer layer disposed on the substrate 212 is “pulled” (interchangeably referred to herein as drawn or elongated) to create fiber-like, elongate protrusions. The fiber-like protrusions (and the viscous polymer layer) are then dried under a drying air flow (either at room temperature or at a slight elevated temperature such as, for example, in a range from about 40° C. to about 80° C.) for a predetermined amount of time so as to solidify the fiber-like protrusions.

Referring back to FIG. 3, in some embodiments, the template mount 202 includes guide posts 238 and spring members 222 positioned concentrically around the guide posts 238. The spring members 222 support the motion of the substrate holder 210 relative to the template mount 202. The spring members 222 provide a restoring force such that in normal condition, the substrate holder 210 is distant from the template mount 202, and more specifically, distant from the contact points 207 of template 201. In some embodiments, during operation, the stamper arm 242 may provide a downward force, compressing the spring members 222, thereby causing the substrate holder 210 to move toward the template 201. The movement of the substrate holder 210 towards the template 201 causes the polymer disposed on the substrate 212 to come in contact with contact points of the template 201. In some embodiments, after a predetermined time of contact between the contact points of the template 201 and the polymer disposed on the substrate 212, the stamper arm 242 may release the downward force in a controlled manner to allow the spring members 222 to move the substrate holder 210 away from the template 201 at a predetermined rate. The movement of the substrate holder 210 away from the template 201 causes the template 201 to “pull” or “draw” microneedles from the polymer disposed on the substrate 212.

Advantageously, the spring members 222 obviate the need for providing upward or pulling force to the substrate holder 210, thereby simplifying the drive assembly 240 used for moving the stamper arm 242. Moreover, because the maximum separation between the template 201 and the substrate holder 210 can be controlled by the spring members 222, the template mount 202 can be moved away from the stamper arm 242 (either by translating the stamper arm 242 or by translating the template mount 202) and replaced with a new template mount 202 while the microstructure formed on the preceding template mount 202 dries. In other words, the spring loaded substrate holder 210 with a stamper arm 242 applying force in one direction enables formation of an assembly line for a high-throughput production of the microstructures such as the patch 100 described above.

In some embodiments, the assembly line structure may be further facilitated by structuring the table 230 to hold a plurality of template mounts 202 which can be translated over different airflow ingress channels 232. In some embodiments, the different airflow ingress channels 232 may have different airflow characteristics depending on, e.g., whether the corresponding template mount 202 is undergoing the “pulling” step or whether a microstructure has already been formed (i.e., the template mount 202 already has a microstructure that is in process of drying). In some embodiments, the table 230 may be mounted to an assembly (not shown) to allow multiple tables 230 to be translated across the airflow ingress channels 232. The tables 230 may further include alignment structures such as, e.g., overhang handles on opposite corners (not shown), to enable the tables 230 to nest together into a continuous line without mechanical interference. In some embodiments, the tables 230 may additionally or optionally include indexing notches to facilitate automated positions relative to the airflow ingress channels 232. The airflow ingress channels 232 may also be fitted with locking structures (not shown) such as, for example, spring detents that reversibly lock the tables 230 over corresponding airflow ingress channels 232.

Advantageously, the present disclosure enables the creation of specific microstructure configurations and as discussed herein, and some embodiments can perform such manufacturing faster and more accurately than previously possible.

### Forced Airflow Mechanism for the Manufacturing Apparatus

As noted above, in some embodiments, the apparatus 200 can optionally comprise an airflow mechanism that directs air during the manufacturing process toward the microstructures to enable a uniform drying and/or solidification of the viscous polymer forming the microstructures. Without wishing to be bound by theory, air flow during solidification of the viscous polymer can provide a faster solidification and a more uniform solidification of the viscous polymer, thereby increasing the manufacturing speed (i.e., throughput), yield, and reliability of the manufacturing process. Further, when testing the microstructure products formed by the methods disclosed herein, metrology confirms the efficacy and consistency of the process. Therefore, given the speed improvements, as well as improvements in the results, substantial cost and time savings can be achieved using the methods disclosed herein.

FIG. 2 illustrates an aspect of the forced airflow system. The apparatus 200 illustrated in FIG. 2 includes both the airflow ingress channels 232 and the inflow hose 234. In some embodiments, the airflow ingress channel 232 facilitates an airflow during the manufacturing of the microstructures.

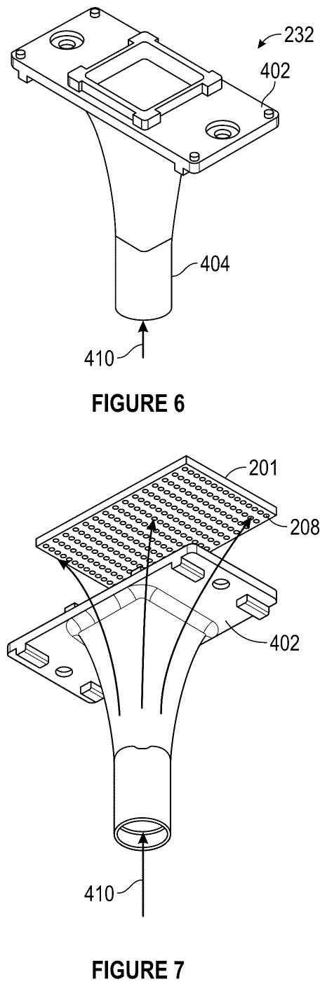

FIGS. 6 and 7 illustrate perspective views of the airflow ingress channel 232, in accordance with some embodiments of the present disclosure. As shown, the airflow ingress channel 232 may include a mount or an attachment plate 402 that can connect the airflow ingress channel 232 to one of the slots 236 of the table 230. The airflow ingress channel 232 can be formed as a hollow channel and have an inflow structure 404 that connects the attachment plate 402 to the air hose 234. In this manner, the inflow end of the airflow ingress channel 232 can be in fluid communication with the outflow and at the attachment plate 402. Therefore, the airflow ingress channel 232 can permit airflow 410 to the template base 204 of the template 201.

In some embodiments, air may be provided to the airflow ingress channel 232 by the air hose 234 (see FIG. 2), to create airflow 410 towards slot 236 and a corresponding stamping assembly 250. Optionally, the stamping assembly 250 can comprise one or more outlet apertures that permit the airflow 410 to be directed toward the substrate 212. In some embodiments, the template itself can comprise outlet apertures for permitting airflow therethrough to be directed toward the substrate.

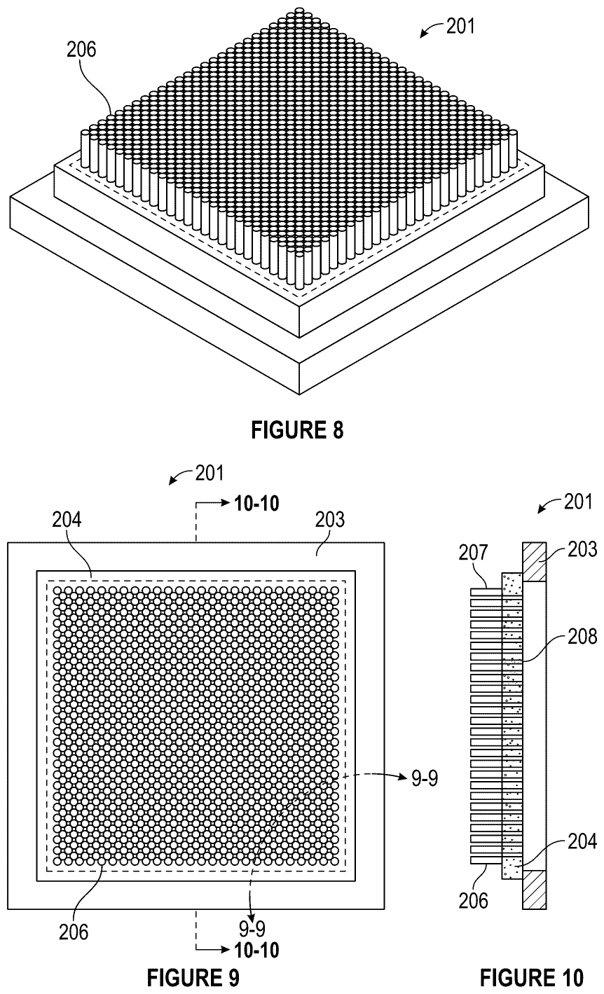

FIG. 8 illustrates a perspective view of the template 201 that can be used for manufacturing the microstructures, in accordance with some embodiments of the present disclosure. An embodiment of the template 201 that can be used for manufacturing the microstructures is shown in a plan view in FIG. 9 and in a side view in FIG. 10, to best illustrate aspects of the template, in accordance with some embodiments of the present disclosure.

### Template Design: Raised Structures

The template may include a textured, raised, or other surface or structure that enables the template to be contacted against a planar surface at a plurality of contact points. These contact points can be formed from raised structures, such as a plurality of pins, bumps, pillars, and/or other structures protruding from the template.

For example, as shown in FIG. 8, the template 201 includes raised structures or points such as, for example, the pins 206 that form a plurality of contact points. These raised structures (e.g., pins 206 or bumps that have been formed by stamping or pressing the template base 204) can be regularly spaced on the template base 204.

In some embodiments of the template 201 that include bumps formed on the template base 204, the bumps may be formed integrally on the surface of the template base 204 (i.e., have the same material as the template base 204) or by externally depositing a different material on the surface of the template base 204 of the template 201. In some embodiments, the externally provided bumps may include a second viscous polymer and a therapeutic agent.

The raised structures can be arranged in any of a variety of arrays or patterns. For example, the raised structures or pins 206 can be arranged in a series of rows, concentric circles, or other such patterns. The arrays can be arranged as square or rectangular arrays, comprising from 10 to 100, from 15 to 50, from 20 to 40, or about 30 contact points per row and from 10 to 100, from 15 to 50, from 20 to 40, or about 30 contact points per column. In some embodiments, the raised structures and/or the arrays can be arranged in a hexagonal configuration. Further, in some embodiments, the arrangement of the raised structures can be modified or prepared by excising certain shapes from a larger array. Other patterns can also be used, with irregular or random spacing of the protrusions, or with specific asymmetric patterns that may be tailored to be appropriate for specific body structures and surfaces desired for treatment. Additionally, multiple patches, including excised patches, can be combined into a larger composite patch, embodying specific needle densities or patterns, as desired.

For example, as shown in FIGS. 8-10, in some embodiments, the template 201 can include an array of pins (e.g., with sharp tips) or pillars (e.g., with flat heads) that extend from the template base 204 of the template 201. In some embodiments, the length (or height) of the pins or pillars 206 can range from about 0.2 mm to about 20 mm, e.g., about 0.5 mm, about 1 mm, about 2 mm, about 5 mm, about 7.5 mm, about 10 mm, about 12.5 mm, about 15 mm, about 17.5 mm, about 20 mm, or any height between any two of these values.

The area, density, spacing, height, base dimensions, contact point dimensions, composition, contact point flatness, etc. attributes of the raised regions of the template 201 are not particularly limited, and can be determined by those skilled in the art based on the particular application for which the microstructure is being manufactured.

Accordingly, the number of raised structures on the template base 204 can be varied in accordance with a desired design or specification of the microstructure to be formed using the template 201. In this manner, the raised structures can serve as the textured or other surface or structure that can contact a surface of the substrate 212 or a polymer disposed thereon.

### Template Design: Airflow Apertures

As noted herein, providing an airflow during the manufacturing process may advantageously increase the uniformity of the airflow, increase the uniformity of drying and solidification of the composition, and decrease the processing time by accelerating the drying and solidification process. These substantial benefits can be achieved using one of the various embodiments disclosed herein.

These benefits and others can be achieved, for example, using airflow from the template directed toward the substrate holder through the outlet apertures in the template.

For example, referring now to FIGS. 8-12, the template 201 can optionally be configured to include one or more airflow apertures that enable airflow to be directed through the template 201 toward the substrate. As illustrated in FIGS. 10-12, the template 201 can include outlet apertures 208 for redirecting the airflow from the airflow ingress channel 232 out through the template 201 and toward the substrate holder 210 and substrate 212 during the manufacturing process.

In some embodiments, as illustrated in FIG. 10, the template 201 can include outlet apertures 208 spaced alternately with the pins (or pillars or bumps) 206, to permit air outflow at the base of each pin 206, facilitating rapid and even drying of the polymer in contact with the plurality of contacts points 207 when the template 201 is brought in contact with a polymer disposed on the substrate 212 and drawn into projections (as discussed in detail elsewhere herein).

The outlet apertures 208 may be formed in the template base 204 that receive air through an air ingress provided at the template holder 202. For example, in some embodiments, air may be provided through the air hose 234, and directed to the template 201 by the airflow ingress channel 232. The air is the pushed through the outlet apertures 208 of the template 201 towards the substrate 212, thereby drying and solidifying the polymer in contact with the plurality of contact points 207 of the template 201. Without wishing to be bound by theory, an outlet aperture 208 between every adjacent pair of pins 206 of the template 201 may facilitate a uniform air flow to the viscous polymer in contact with the pins 206.

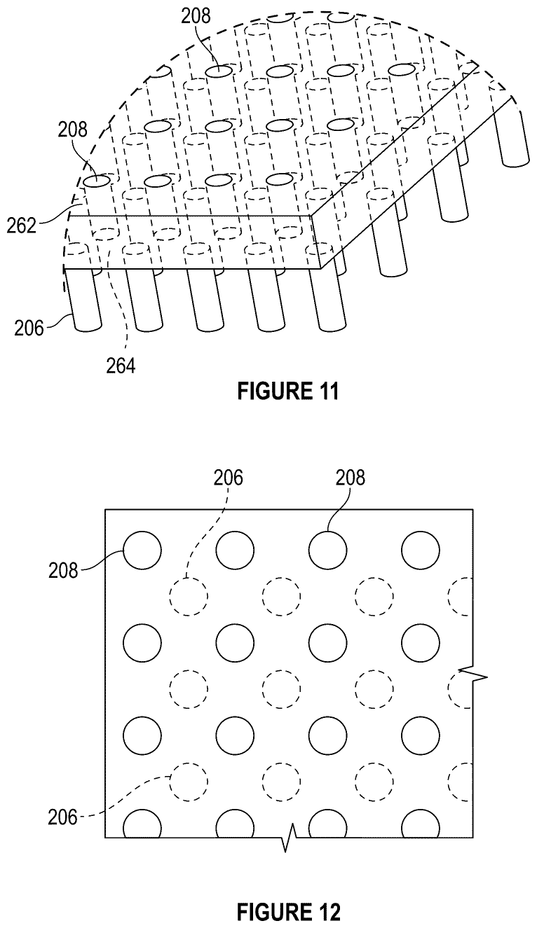

FIGS. 11 and 12 illustrate an enlarged, detail view of the template 201 showing the outlet apertures 208 provided in the template base 204. In some embodiments, as shown in FIGS. 9 and 10, the outlet apertures 208 are positioned between every adjacent pair of pins 206 of the template 201 and extend from a first surface 262 of the template base 204 to a second opposing surface 264 of the template base 204. The first surface 262 in some embodiments is disposed on the template holder 202 such that the pins 206 extend from the second surface 264. Thus, the outlet apertures 208 in some embodiments provide a path for flow of air provided through the airflow ingress channel provided at the template holder 202 to the base of each of the pins 206.

The size, density, spacing, height, base dimensions, contact point dimensions, composition, contact point flatness, etc. attributes of the outlet apertures 208 of the template 201 are not particularly limited, and can be determined by those skilled in the art based on the particular application for which the microstructure is being manufactured.

### Manufacture of the Microstructures

In another aspect of the present disclosure, methods of manufacturing a microstructure are described herein. The methods described herein result in microstructures that are continuous with the layer over which the microstructures are formed, thereby increasing the strength of the individual microstructures. Additionally, the method allows high-throughput and reliable manufacturing of the microstructure using, e.g., the apparatus 200 described herein. Moreover, as further described in detail herein, because a continuous film of a viscous polymer is contacted with a template, a need for aligning the template with the viscous polymer is eliminated, further increasing the throughput of the process.

FIGS. 14-22 illustrate the apparatus 200 for manufacturing the microstructure during various stages of performing a method of manufacturing the microstructure, in accordance with some embodiments of the present disclosure.

In some embodiments, a method of manufacturing a microstructure can include depositing a layer 216 of composition, such as a viscous polymer, onto a substrate 212 and contacting a template 201 having a plurality of contact points 207 against a surface of the viscous polymer layer 216. The template 201 can be separated from the surface of the viscous polymer layer 216 to draw the viscous polymer into a plurality of projections 218. The projections 218 are then permitted to solidify.

FIG. 14 shows an initial configuration where the template 201 and the substrate 212 are spaced apart such that there is a gap between the contact points 207 of the template 201 and the surface of the polymer layer 216. In some embodiments, contacting the contact points 207 of the template against the surface of the viscous polymer layer 216 (also referred to herein as “needle-forming layer” or a “microneedle-forming layer”) may include moving the substrate 212 relative to the template 201 vertically, as illustrated in FIG. 15, to bring the contact points 207 in contact with the surface of the viscous polymer layer 216, as illustrated in FIG. 15.

In some embodiments, a certain amount of time may be allowed to pass before separating the template 201 from the substrate 212 so as to form the microneedles. Such time may allow localized diffusion of the therapeutic agent into the viscous polymer so that the microstructure formed using such a template are formed loaded with the therapeutic agent. The amount of time for which the template 201 remains in contact with the substrate 212 before being separated may range from 1 second to a few minutes. For example, the time may be 1 seconds, 5 seconds, 10 seconds, 15 seconds, 20 seconds, 25 seconds, 30 seconds, 40 seconds, 50 seconds, 1 minute, 1.25 minutes, 1.5 minutes, 1.75 minutes, 2 minutes, 2.5 minutes, 3 minutes, 3.5 minutes, 4 minutes, 4.5 minutes, 5 minutes, 6 minutes, 7 minutes, 8 minutes, 9 minutes, 10 minutes, or any amount of time between any two of these values. It will be appreciated that the amount of time provided herein may be approximate within the tolerance limits of the manufacturing process. For example, the time may deviate from an intended time because of a number of factors related to the manufacturing process such as, for example, a delay in activation of the various actuators, or a delay in sensing that the template 201 and the substrate 212 are in contact. Thus, a deviation about 10% from the intended value is contemplated within the scope of the disclosure.

In some embodiments, the pins or pillars 206 of the template 201 may be provided with a second viscous polymer and/or a therapeutic agent. For example, the template 201 can be contacted with a layer or reservoir of the second viscous polymer in which the therapeutic agent has been dispersed prior to contacting the template 201 with the viscous polymer layer disposed on the surface of the substrate 212 on which the microstructure is to be formed. In such embodiments, the therapeutic agent may diffuse into the viscous polymer during the formation of the microneedles such that the tips of the microneedles formed using such “coated” pin/pillar array are formed loaded with the therapeutic agent.

Any suitable substrate 212 may be used for manufacturing the microstructures. Suitable substrates may have certain properties that enable appropriate handling and manufacturing tolerances. For example, suitable substrates may have an appropriate combination of mechanical rigidity, thickness, flatness and surface finish.

In some embodiments, the manufacturer of the microstructures can provide for a computer-executed program that accounts for substrate thicknesses, compositional layer thicknesses, and desired microstructure dimensions, such as length and thickness. An accurate measurement of thickness enables handling of the substrate and subsequently manufactured devices as well as knowledge of the position of an upper surface of the substrate during the manufacturing process.

For example, an accurate measure of layer thicknesses can enable the system to precisely control the movement of the components of the apparatus and precisely position the composition disposed on the substrate relative to the raised structures of the template. Such precise computer or manual control can facilitate movement of the upper surface of the substrate relative to a mounting surface during the manufacturing process, with known layer thicknesses enabling the system to understand and move the substrate to a position in which the mounting surface is in contact with the upper or lower surface of the substrate. Further, the system can employ any of a variety of optical sensors to control movement of its components during the manufacturing procedure.

For example, in accordance with some embodiments disclosed herein is the realization that if the substrate 212 is placed so that the bottom surface is in contact with a piece of equipment, the position of the top surface of the substrate 212 can be precisely defined, permitting, for example, the thickness of a polymer layer spread across the top surface to be controlled to the same precision. Further, the thickness of the substrate may impact the relative rigidity, weight, heat-capacity, and other physical characteristics of the substrate, and variations in these properties within a population of substrates can impact quality by increasing variability in the processing environment experienced by each polymer film produced. In order to account for such variations, the system can “learn” the properties of the substrates and compositions applied thereto using any of a variety of sensors and develop a suitable program or process that accounts for variation in such parameters. Additional suitable methods of casting polymers are described in U.S. Publication No. 2016/0279401, which is herein incorporated by reference in its entirety.

Further Aspects of Microstructure Manufacturing Processes and Systems, and their Products

In addition to other details and aspects of the methods disclosed herein, the inventors of the present technology have also made certain novel and inventive realizations regarding the processes, equipment, and products disclosed herein.

For example, one of the aspects in accordance with some embodiments is that suitable substrates should have an appropriate amount or level of flatness. Appropriate amount/level of flatness may enable uniform spreading of the viscous polymer layer when disposed on the substrate. Moreover, variation in flatness may adversely affect the uniformity of the size of the microstructures being manufactured by causing variation in points of contact between the surface of the viscous polymer and the points of contact of the template. Similarly, variation in flatness may also result in misregistration and instability of the substrate relative to the mounting substrate.

In accordance with some embodiments, an appropriate surface finish of the substrate may reflect a combination of surface properties such as, for example, roughness, waviness, reflectivity and other aspects related to the microscopic topology of the substrate surface. One aspect that can be strongly dependent upon the surface finish is the tendency of the polymer solution film to adhere to the substrate, or conversely to peel away from the substrate during and after the process of drying the layer of the viscous polymer. For instance, a mirror-like surface (i.e., a very smooth, reflective surface finish) may not provide sufficient roughness to adequately anchor the drying polymer film, resulting in spontaneous peeling, while an overly grainy, rough surface tends to promote over-adhesion such that removal of the film requires such force that the microneedle arrays may be damaged.

Additionally, in accordance with some embodiments, surface finish can impact cleanliness, and excessive pitting can be undesirable in that such pockets could harbor bacteria and hinder removal of contaminants. Further, the reflectivity of the substrate may have a strong effect upon optical imaging of the microneedle structures formed upon the surface, and a mirror-like, highly reflective surface may make the imaging for quality analysis and inspection difficult. Some surface roughness can be desirable in order to provide a visually grainy background for imaging of the overlying transparent layer of the viscous polymer.

In accordance with some embodiments, suitable substrates can be of any appropriate solid or porous material onto which a polymer solution can be applied such as, for example, glass, quartz, steel, copper, backing layer materials including woven and non-woven material, polymethylmethacrylate (PMMA), etc. The thickness of the substrate may be, for example, in a range from about 0.1 inches to about 1.5 inches, with a flatness in a range from about 0.001 inches to about 0.05 inches. In some embodiments, the substrate can have a thickness of 0.7±0.002 inches. Similarly, the substrate may have a surface roughness of about N16 or smoother. For example, in some embodiments, the substrate may have a surface roughness of about N8 (i.e., average profile roughness of about 125 pin (or 3.2 μm).

### Compositions for Microneedle Products

As disclosed herein, some embodiments can provide a composition that is useful to be drawn into a microneedle product. The composition can comprise a viscous polymer suitable for such applications, and optionally, one or more drugs or active agents.

The term “viscous polymer” used herein can refer to a composition that contains a viscous material. The viscosity of the viscous polymer may be appropriately adjusted by changing the kinds, concentrations, and temperature of a viscous material and other materials contained in the viscous polymer or by adding a viscosity modifier. Although the viscosity of the viscous polymer may not be limited to a particular value, in some embodiments, the viscosity can be 200,000 cSt or less.

An example of the viscous material that can be contained in the viscous polymer is a cellulose polymer such as, e.g., hydroxypropyl methylcellulose, hydroxyalkyl cellulose (preferably, hydroxyethyl cellulose or hydroxypropyl cellulose), ethyl hydroxyethyl cellulose, alkyl cellulose, and carboxymethylcellulose. Non-limiting examples of the viscosity modifier may include hyaluronic acid and salts thereof, polyvinylpyrrolidone (PVP), cellulose polymer, dextran, gelatin, glycerin, polyethylene glycol, polysorbate, propylene glycol, povidone, carbomer, gum ghatti, guar gum, glucomannan, glucosamine, dammer resin, rennet casein, locust bean gum, microfibrillated cellulose, psyllium seedgum, xanthan gum, arabino galactan, gum arabic, alginic acid, gelatin, gellan gum, carrageenan, karaya gum, curdlan, chitosan, chitin, tara gum, tamarind gum, tragacanthgum, furcelleran, pectin, or pullulan.

In some embodiments, the viscous polymer may contain only a viscous material. In some embodiments, the viscous polymer can further include at least one active ingredient such as, for example, a therapeutic agent. In some embodiments, the active ingredient includes drug molecules or biomolecules (i.e., biological entities). In some embodiments, the active ingredient comprises an antigen, antibody, or toxin. In still some embodiments, the active ingredient is a neurotoxin such as a botulinum toxin, for example. Botulinum toxin of types A, B, C, D and/or E can be present in the microneedle arrays. In some embodiments, the botulinum toxin is selected from the group consisting of Botulinum toxin serotype A (BoNT/A), Botulinum toxin serotype B (BoNT/B), Botulinum toxin serotype C1 (BoNT/C1), Botulinum toxin serotype D (BoNT/D), Botulinum toxin serotype E (BoNT/E), Botulinum toxin serotype F (BoNT/F), Botulinum toxin serotype G (BoNT/G), Botulinum toxin serotype H (BoNT/H), Botulinum toxin serotype X (BoNT/X), Botulinum toxin serotype J (BoNT/J), and mosaic Botulinum toxins and/or variants thereof. Examples of mosaic toxins include BoNT/DC, BoNT/CD, and BoNT/FA. In some embodiments, the botulinum toxin can be a sub-type of any of the foregoing botulinum toxins. Other suitable therapeutic agents that can be used in conjunction with the microstructures disclosed herein are discussed in U.S. Publication No. 2018/0236215 which is incorporated herein by reference in its entirety.

The viscous polymer, in some embodiments, may further contain at least one biocompatible material and/or a biodegradable material. The term “biocompatible material” can refer to a material that is substantially non-toxic in a human body, chemically inactive, and deficient in immunogenicity. The term “biodegradable material” can refer to a material that is degradable by body fluids or microorganisms in living bodies. The biocompatible or biodegradable material serves as a skeletal material of microstructures according to the present invention.

Non-limiting examples of the biocompatible material and/or biodegradable material may include polyester, polyhydroxyalkanoates (PHAs), poly(α-hydroxy acid), poly(β-hydroxy acid), poly(3-hydroxybutyrate-co-valerate) (PHBV), poly(3-hydroxy proprionate) (PHP), poly(3-hydroxyhexanoate) (PHH), poly(4-hydroxy acid), poly(4-hydroxybutyrate), poly(4-hydroxyvalerate), poly(4-hydroxyhexanoate), poly(ester amide), polycaprolactone, polylactide, polyglycolide, poly(lactide-co-glycolide) (PLGA), polydioxanone, poly(ortho ester), polyetherester, polyanhydride, poly(glycolic acid-co-trimethylene carbonate), polyphosphoester (PPE), PPE urethane, poly(amino acid), polycyanoacrylate, poly(trimethylene carbonate), poly(iminocarbonate), poly(tyrosine carbonate), polycarbonate (PC), poly(tyrosine arylate), polyalkylene oxalate, polyphosphazene, PHA-PEG, ethylenevinyl alcohol copolymer (EVOH), polyurethane, silicon, polyester, polyolefin, polyisobutylene-ethylene-α-olefin copolymer, stylene-isobutylene-stylene triblockcopolymer, acryl polymers and copolymers, vinyl halide polymers and copolymers, polyvinyl chloride, polyvinyl ether, polyvinyl methylether, polyvinylidene halide, polyvinylidene fluoride, polyvinylidene chloride, polyfluoroalkene, polyperfluoroalkene, polyacrylonitrile, polyvinyl ketone, polyvinyl aromatics, polystyrene, polyvinyl ester, polyvinyl acetate, ethylene-methyl methacrylate copolymers, acrylonitrile-stylene copolymer, polyvinyl alcohol (PVA), polyacrylates, polymers of ethylene-vinyl acetates, and other acyl substituted cellulose acetates, polyurethanes, polystyrenes, polyvinyl fluoride, polyethylene oxide, chlorosulphonate polyolefins, poly(vinyl imidazole), poly(valeric acid), poly butyric acid, poly lactides, polyglycolides, polyanhydrides, polyorthoesters, polysaccharides, gelatin, ABS resin-ethylene-vinyl acetate copolymer, polyamide, alkyde resin, polyoxymethylene, polyimide, polyether, polyacrylate, polymethacrylate, polyacrylic acid-co-maleic acid, chitosan, dextran, cellulose, heparin, hyaluronic acid, alginate, inulin, starch, glycogen and the like, mixtures, and copolymers thereof.

The biocompatible material or biodegradable material can have a certain level of viscosity when it is dissolved in a solvent. Examples of the solvent may include, but not limited to, water, absolute or hydrous lower alcohol having 1 to 4 carbon atoms, acetone, ethyl acetate, chloroform, 1,3-butylene glycol, hexane, diethyl ether, and butylacetate.

For example, in some embodiments, PVA solutions are used. A composition using a specific PVA polymer raw material (Emprove™ 40-88, USP, MilliporeSigma, Darmstadt, Germany) comprises high-purity, pharmaceutical grade, low-endotoxin polymer of a nominal average molecular weight in a range from about 50 kDa to about 5000 kDa, e.g., about 75 kDa, about 100 kDa, about 150 kDa, about 200 kDa, about 500 kDa, about 750 kDa, about 1000 kDa, about 2500 kDa, about 4000 kDa, or any molecular weight between any two of these values. Advantageously, this material produces solutions with appropriate rheological properties to efficiently produce optimally-shaped microneedles using the methods described herein. Use of a lower molecular weight grade of PVA may result in needles of incorrect/inferior morphology and reduces the yield of viable microneedle devices thus produced.

In some embodiments, a layer of PVA solution can be spread directly onto a steel substrate, and needle structures are formed directly from that layer, with no intervening layers or drying steps. The polymer solution used for this single-layer embodiment may be between 25-35% PVA, and for some embodiments, preferably 30%. If the polymer solution concentration is too low, thin needles will result, and fiber-like intermediate structures will tend to rupture prematurely, not forming useful needles. If the polymer solution is too high, bridging between adjacent needles may frequently occur, causing deformation and conjoined needle structures that are not useful, reducing the production yield.

It must be noted that production of uniform aqueous solutions containing 20% or more PVA polymer may be non-trivial. For example, it was found that in some instances, the material required substantial energy input in the form of temperature and shear, and further was highly prone to entrainment of air, forming bubbles that may be difficult to remove from the resulting high-viscosity solution.

Further, dissolution and full hydration of the long polymer chains may take substantial time, and incomplete or uneven processing may result in inhomogeneous solution product, with lumps or inclusions of incompletely dissolved or dispersed material. The presence of bubbles and/or lumps both create localized inhomogeneity in the polymer films utilized in the methods described herein, and produce either point-defects in which one or more needles in that region are compromised, or in the case of a lump dragged across the film during spreading, leaving a trench of non-uniform thickness, the entire film area can be compromised. Thus, the method of processing PVA to obtain a solution of requisite molecular weight and wt. % is also disclosed herein.

For example, in some embodiments, PVA having the requisite molecular weight is mixed with water in requisite ratio using a centrifugal mixer, which uses centrifugal force generated by a rotor arm to exert shear in an independently rotating off-axis process cup, positioned at the end of the rotor arm, which can itself be swinging the rotating cup in a continuous arc. The process cup may be heated, e.g., in a common microwave oven, and may contain both the granular PVA polymer material and the water used to form the solution. In some embodiments, the solution can be briefly boiled, promptly mixed at, a predetermined rpm for a predetermined amount of time (based on parameters such as the molecular weight of the polymer, the amount of solvent, other additives, etc.), and then left for several hours, during which time the exterior channel ions of the polymer granules hydrate further. After this hydration “rest” period, the process can be repeated, to disperse and incorporate the hydrated polymer into the solution bulk. Gradually, e.g., through 5-10 such cycles, the granules may be hydrated and eroded to the point of complete incorporation into a homogeneous polymer solution.

In some embodiments, the method for obtaining a PVA solution described herein, however, may generate many fine bubbles, due both to entrainment in the granular polymer raw material and to the heating cycles, which may produce localized boiling in portions of the polymer mixture, especially around incompletely dissolved granules. It is noted that generally bubbles in the highly viscous resulting solutions do not spontaneously clear by buoyancy in useful time periods, especially fine bubbles, which will remain suspended for days or weeks without further processing. Therefore, the solutions produced by this method, in some embodiments, are heated to above 50° C., and centrifuged for at least several hours, or until clear. Centrifugation at 1000×g for 3 hr at 40° C. (produces a solution with high clarity, showing few if any entrained bubbles over about 100 μm in diameter. In some embodiments, the solutions are kept under vacuum for a certain period of time to allow degassing.

In some embodiments, a programmable mixer with a paddle agitator can be used to slowly stir the mixture of PVA and water at a process temperature of about 90° C. for several hours until uniform. Without wishing to be bound by theory, by use of a sufficiently slow stirring speed (low enough that bubbles are not entrained into the solution) the solution can be produced with sufficiently low bubble content that a centrifugation or degassing step may not be necessary.

In some embodiments, the viscous polymer further can include plasticizers. Plasticizers can improve adhesion between the substrate and the polymer layer and can further improve compatibility between two polymer layers if multiple layers of polymer(s) are used. Examples of plasticizers include, but are not limited to, polyethylene glycol, glycerin, and citrate esters.

In some embodiments, the plasticizer can include polyethylene glycol 400 and triethyl citrate. Plasticizers make the polymer more flexible in dried form, such that the interfacial stresses that develop between the rigid substrate and the drying polymer solution, which contracts as it dries, can be relieved by stretching and similar deformation of the polymer, making it less prone to out-of-plane deformation that can result in premature or spontaneous peeling or adhesion loss. However, the flexibility can also make the microneedle structures themselves more prone to flexion, compromising skin penetration during application. Therefore, useful ranges of plasticizers are typically low, under 1% content.

In some embodiments, the viscous polymer can include polyvinyl alcohol (PVA). In some embodiments, the viscous polymer can include or sodium hyaluronate or hyaluronic acid (both referred to herein as HA).

### Preparation of the Substrate for Manufacturing

Aspects of the manufacturing methods are recited generally herein and include contacting a plurality of raised structures against a composition disposed on a substrate. Aspects of preparation and placement of the composition on the substrate will now be described.

Any suitable method of disposing the viscous polymer on the substrate may be used so long as it provides a generally continuous layer with a uniform thickness. For example, in some embodiments, the viscous polymer can be disposed on the substrate using spin coating, wherein a certain amount of viscous polymer can be poured on the substrate and the substrate can be spun at a certain RPM. The RPM typically determines the thickness of the layer. In some embodiments, the substrate may be placed on a magnetic or a vacuum chuck or substrate carrier so as to immobilize the substrate during the spinning.

In some embodiments, the viscous polymer can be poured on a leveled substrate and allowed to spread under gravity. In some embodiments, a certain amount of viscous polymer can be poured on the substrate and a layer of desired thickness can be obtained by sliding a single-edged razor blade across the substrate at a certain separation.

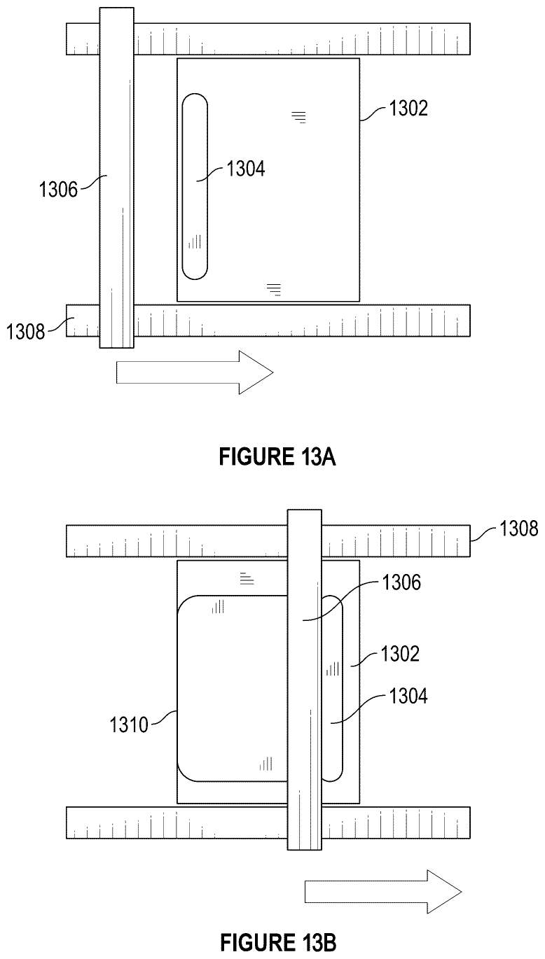

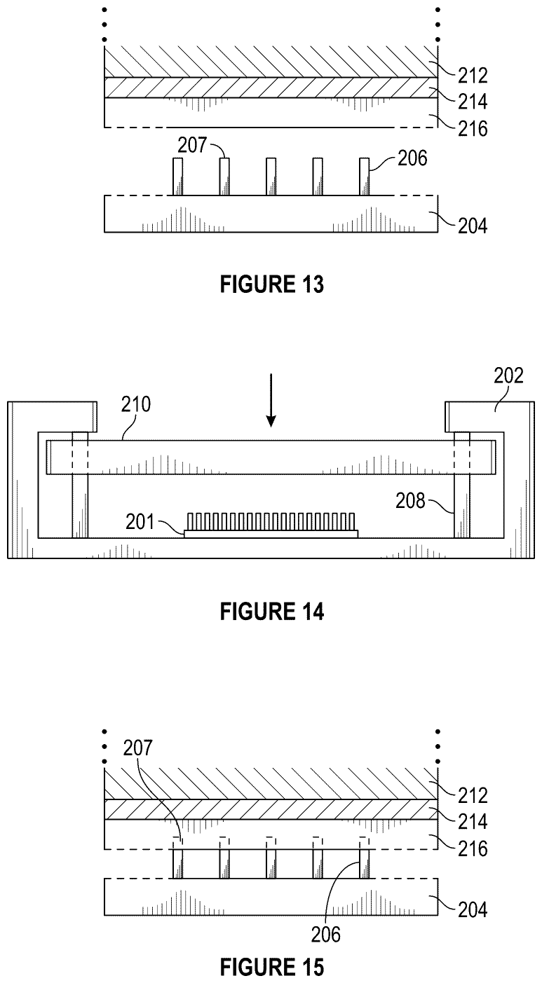

FIGS. 13A and 13B illustrate a method of forming a continuous layer of a viscous polymer with uniform thickness in accordance with some embodiments. In at least one embodiment, the viscous polymer is poured on a leveled substrate 1302, and a bar (also referred to herein as a “draw bar”) 1306 is drawn across the leveled substrate 1302. The bar 1306 may be a precision ground cylinder in an embodiment. In some embodiments, the bar may have other cross-sections such as, a rectangle, an I-beam or other suitable shapes.

A precise gap may be maintained between the draw bar 1306 and the substrate 1302 while the draw bar 1306 is drawn across the substrate 1302 so as to spread the viscous polymer on the substrate 1302 at a uniform thickness. The gap between the draw bar 1306 and the substrate 1302 may be set by leveling the substrate 1302 relative to two parallel rails 1308 flanking the substrate 1302. It will be appreciated that the gap between the draw bar 1306 and the substrate 1302 determines the thickness of the continuous layer 1310 of the viscous polymer, and as such, the gap may be set based on the desired thickness of the continuous layer 1310.

A stripe 1304 of the viscous polymer may then be disposed on the substrate 1302 at an amount sufficient to form the continuous layer 1310. The draw bar 1306 may be dragged or drawn across the substrate 1302 along the rails 1308 as indicated by the arrow in FIGS. 13A and 13B. The rate at which the draw bar 1306 is drawn across the substrate 1302 may be dependent on factors such as the viscosity of the polymer, the shape of the draw bar 1306 the desired thickness of the continuous layer 1310 and the properties of the underlying substrate 1302 (e.g., wettability of the material of the substrate 1302 by the polymer).

In at least one embodiment, the draw bar 1306 is biased to maintain a contact with the rails 1308 using, for example, a spring-loaded mechanism (not explicitly shown). Such a bias may be advantageous in ensuring that the gap between the draw bar 1306 and the substrate 1302 remains constant, thereby tightly controlling the thickness of the continuous layer 1310.

In some embodiments, a degassing step may be needed after the layer of the viscous polymer is formed on the substrate to ensure that no air bubbles remain in the layer.

In some embodiments, as shown generally in FIGS. 14, 16, 18, 20, and 21, a second polymer layer 214, interchangeably referred to herein as an intermediate layer or a release layer, can be provided between the substrate 212 and the needle-forming layer 216 of the viscous polymer. In accordance with some embodiments, the release layer 214 can improve adhesion or conversely permit clean separation of the overlying viscous polymer layer used for microstructure formation.

For example, in some embodiments, the release layer 214 can be formed of the same polymer as the microneedle layer 216, while in some embodiments, it can be a different polymer. If a different polymer is used as a release layer, that polymer may be water-soluble, as the microneedle-forming layer, or it can be non-water soluble. Examples of water-soluble layers that can be useful as release layers include carboxymethylcellulose and polyvinyl alcohol, or any other water-soluble polymer that dries to form an adherent layer on the underlying substrate (and may further comprise a plasticizer to promote such adhesion). In some embodiments, the release layer 214 comprises polyvinyl alcohol. In some embodiments, the PVA can be applied as a solution between 20% and 30% by weight.

The release layer 214 may or may not remain adherent to the upper microneedle-forming polymer layer 216, and such adherence may not necessarily be a requirement for the release layer 214 to be useful, although one may be selected so as to specifically remain adherent or not, depending upon the desired final product composition. Examples of non-water soluble layers include ethylcellulose, and most particularly a solution of 11% Aqualon EC-N100 ethyl cellulose 11% Aqualon EC-N50 ethyl cellulose (both obtained from Ashland Specialty Ingredients, Covington, KY), 10% water, 3% triethyl citrate (Jungbunzlauer, Ladenburg, Germany), and 0.1% glycerin (J.T. Baker, Phillipsburg, NJ) in ethanol (quantity sufficient to 100%).

In embodiments including the release layer 214, the release layer 214 can be spread over the substrate, dried, and then the needle-forming layer 216 can be spread over the top of the dried release layer 214. The release layer 214 can typically be a different composition compared to the needle-forming layer 216.

Advantageously, for example, in some embodiments, an array of microneedles can be formed by drawing needles from an HA solution that is spread as a needle-forming layer 216 over a dried PVA film 214 (as the release layer) adherent to a stainless steel substrate 212. Without the PVA layer, the HA may spontaneously peels off during drying. The PVA layer may make the HA stay attached during drying to produce a highly planar microneedle product. On the other hand, if the HA/PVA composition described above is removed without separating the films, the underlying PVA film may make the microneedle patches much more rigid. However, the dried HA sheet may be separated from the PVA, as a relatively much more flexible product, which has obvious significant benefits and advantages that have not hitherto been achieved for this technology.

In some embodiments, the microneedle-forming layer 216 can comprise sodium hyaluronate or hyaluronic acid or other soluble salts of HA. In some embodiments, an aqueous solution comprising from about 10% to about 50% wt HA, from about 10% to about 45% wt, from about 15% to about 45%, from about 20% to about 45%, from about 10% to about 40%, from about 15% to about 40%, or any value within any of these ranges, can be spread into a film on a substrate 212 and further processed in accordance with the method of the present disclosure to form the microneedle structures.

### Microstructure Films Using Release Layers

The present disclosure also contemplates the optional use of a release layer during the manufacturing process. Any of a variety of materials can be used as a release layer, which can be interposed between the microstructure composition and the substrate during the substrate preparation steps of the manufacturing process.

In accordance with some embodiments disclosed herein is the realization that HA solutions can contract as they dry and normally do not adhere strongly to a steel substrate surface used in certain embodiments. Such poorly adherent films may, therefore, be prone to spontaneous and/or premature delamination from steel substrates leading to non-planar and out-of-specification microneedle array devices. However, in some embodiments of the present disclosure, unexpectedly and advantageously, the HA solutions described herein readily spread and dry over a previously spread and dried PVA film (i.e., release layer or intermediate layer 214), wetting evenly and not rapidly dissolving the PVA layer.

Advantageously, in accordance with some embodiments disclosed herein, it was discovered that while PVA can be soluble in water, the hydration time is sufficiently long that an HA layer may be applied without fully solubilizing the PVA. Also surprisingly, in some embodiments, although the PVA layer shows signs of hydration, the loss of water from the overlying HA layer does not make it too viscous to spread evenly. Thus, while one of ordinary skill in the art may expect that either the PVA or the HA layer might be disrupted by the presence of the other material, especially with one in dry form and one in wet form, it was found that the HA layer can be evenly and regularly spread over the dried PVA layer, and will in fact dry to form a second, smooth, even, and planar layer that does not spontaneously peel off as it does from a steel substrate in the absence of the PVA layer.

Accordingly, use of a PVA release layer underneath the HA layer was found to be useful, in certain embodiments, to provide an enhancement of adhesion between the HA layer and the underlying steel substrate. This unique and surprising, unexpected result produces advantages and benefits superior to those available in prior microstructure manufacturing techniques and products.

Additionally, and advantageously, it was also observed that in some embodiments, the HA and PVA layers remain attached to the steel substrate without spontaneous peeling even when dried to an extreme dry state in a heated vacuum oven (for example 24 h drying at −18 in Hg vacuum at 40° C.). The layers do not peel prematurely, but can be removed as a single (dual layer) film from the steel with minimal force if separated using, for example, an edge of a razor blade to lift one corner from the steel surface. The layers can then be gently lifted using only light finger pressure to separate them from the steel, and remain planar after removal, so long as they are not given prolonged exposure to a humidity source. For example, in some embodiments, room temperature handling for an hour under typical 30%-60% relative humidity at 22° C.-26° C. does not produce curling, although several days' exposure to these conditions will typically generate substantial curling. Storage using a desiccant packet in a closed container may prevent noticeable curling indefinitely.

Further, it was unexpectedly observed that, in some embodiments, an HA layer can be readily separated from a PVA layer with similar light finger pressure after lifting one edge of the film. Thus, if the razor separation described herein is performed to lift one edge of only the HA layer, leaving the PVA layer adherent to the underlying steel surface, the HA layer alone will readily peel off of the PVA film without removing the PVA from the steel surface. Without wishing to be bound by theory, it is noted that while the PVA can be hydrated by contact with the HA layer, and in fact shows changes in surface reflectivity consistent with hydration or partial solubilization, the polymer layers from the separate layers do not strongly interdigitate to form a strong interface, but rather remain as largely discrete layers even through the wetting and drying cycle of applying and drying the HA film.

Additionally, if the HA film is applied on top of the PVA film as described herein, but overlaps the edge of the PVA film to make contact with the underlying steel substrate, absent any underlying intermediate PVA layer, the HA in contact with the steel will spontaneously peel during drying, and further will delaminate the edge of the underlying PVA film, such that the entire dual film structure will spontaneously delaminate from the underlying steel substrate. In some embodiments, once the out-of-plane deformation of the drying HA layer is commenced, the out-of-plane forces associated with the film curling during drying are substantial enough to overcome the adherence of the PVA to the underlying steel.

Interestingly, as described elsewhere herein in discussing laser processing of these dual films, if the overlying HA layer is cut, as by a laser, it may be done to form a shallow cut that does not fully penetrate and sever the underlying PVA layer. Further, in some embodiments, if the PVA layer is mechanically flexed out of plane after removal from the steel substrate, the HA layer spontaneously delaminates from the PVA, providing an unexpectedly convenient method by which to separate the thin layers that does not require direct contact as by lifting the edge with a razor blade. In some embodiments, the HA layer can be removed or delaminated from the PVA layer by applying lateral shear forces by any of a number of means, including for example airflow, pressure differential, or contact methods.

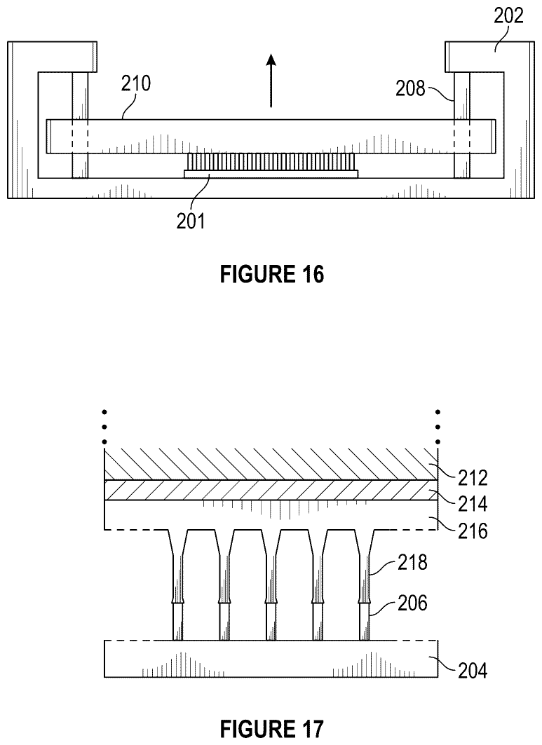

Once the contact points 207 of the pins 206 of the template 201 are contacted with the surface of the viscous polymer 216, the template 201 can be separated by moving the substrate holder 210 vertically away from the template 201, as illustrated in FIG. 16. Such movement results in a separation of the template 201 from the surface of the viscous polymer layer 216 so as to draw the viscous polymer into plurality of projections 218, e.g., fiber like structures extending between the surface of the viscous polymer layer 216 and the contact points 207 of the template 201, as illustrated in FIG. 17.

It is noted that in some embodiments, the separation can be achieved either by moving the substrate 212 on which the viscous polymer is disposed relative to the template 201 or by moving the template 201 relative to the substrate 212. The rate at which the template 201 and the substrate 212 are moved relative to each other determines the geometry and morphology of the microstructure formed. In some embodiments, the rate of separation ranges from about 2 mm/minute to about 50 mm/minute. The rate selected for making a particular morphology of microstructure, e.g., microneedles, can be dependent on the desired morphology as well as the composition of the viscous polymer and the air flow conditions.

For example, for embodiments using PVA as the viscous polymer, separating the substrate and the template at a rate of about 20 mm/minute results in fibers having a length in a range from about 1.5 mm to about 5 mm (depending on the exact composition and viscosity of PVA). Similarly, for embodiments using HA as the viscous polymer, separating the substrate and the template at a rate in a range from about 2 mm/min to about 5 mm/min results in fibers having a length in a range from about 2.5 mm to about 5.5 mm.

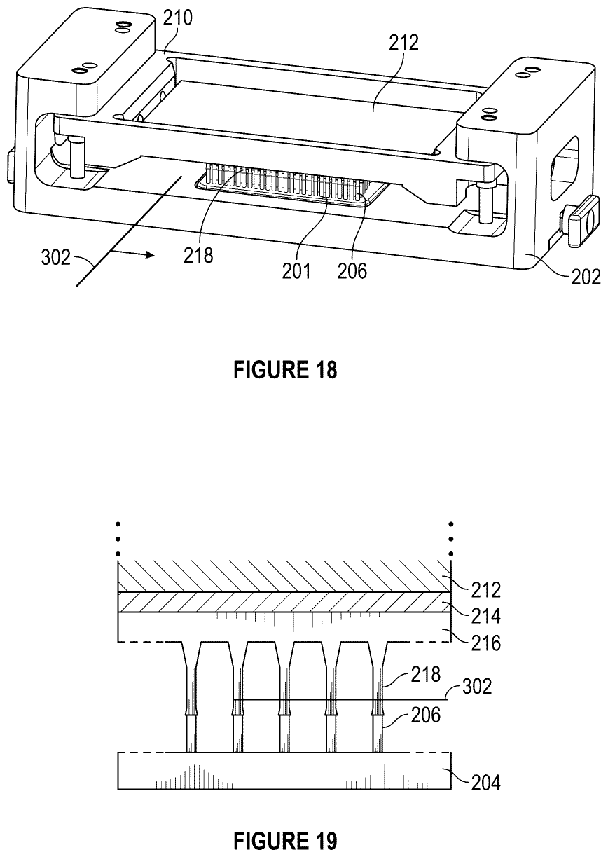

As illustrated in FIG. 18, the projections 218, i.e., fibers, thus formed are then permitted to solidify. As disclosed herein, the solidification process may, in some embodiments, be accelerated by providing an air flow across the projections 218. In embodiments where the air flow is facilitated via outlet apertures (e.g., outlet apertures 208) provided in the template base 204 of the template 201, a more uniform pressure distribution may be obtained across the template 201, thereby improving the uniformity of the morphology of formed projections (i.e., fibers). In some embodiments, the air flow passing through the outlet apertures to reach the viscous polymer layer can be about 0.5 cfm. In some embodiments, heat may also be applied in addition to or in lieu of the air flow to aid or accelerate solidification of the projections.

### Processing of Microstructure Products

After the microstructure has been formed using the drawing techniques disclosed herein, thereby creating a plurality of fiber-like, elongate protrusions, these elongate protrusions can be separated from the raised structures of the template. In some embodiments, as disclosed herein, the substrate and the template can be maintained at a specific separation distance while the fiber-like, elongate protrusions solidify. Thereafter, the fiber-like, elongate protrusions can be cut or broken at a desired point along their length in order to form the microstructure.

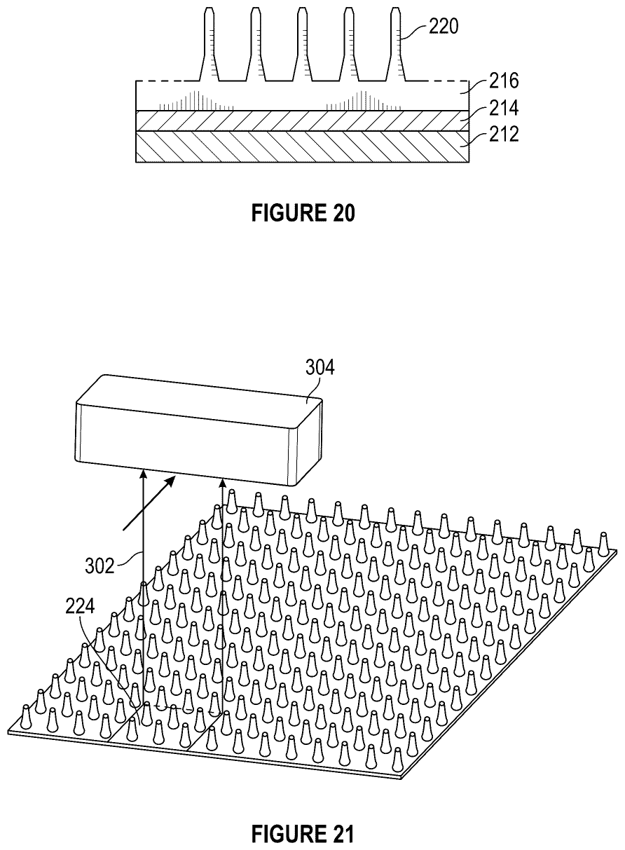

For example, once the projections 218 extending from the contact points 207 of the template 201 to the surface of the viscous polymer layer 216 are formed and solidified, the projections 218 may be cut, as illustrated in FIGS. 19 and 20, at a desired height (from the surface of the viscous polymer) to form the desired microstructure illustrated in FIG. 19. Typically, the microstructure formed upon cutting the projections 218 can be an array of microneedles 220. Depending on the exact process used to form such array of microneedles 220, the individual microneedles 220 may or may not include a therapeutic agent. The microneedles 220 may have a length in a range from about 10 μm to about 2070 μm.

Any suitable method may be used for cutting the projections 218 to form the microneedles 220.

For example, in some embodiments, the cutting of the projections 218 can be performed in a manner in which the template 201 and the substrate 212 on which the viscous polymer is disposed are moved laterally relative to each other to break the projections.

In some embodiments, the cutting of the projections 218 can be performed in a manner in which an ultrasound pulse can be applied to either the template 201 or the substrate 212 (or both) so as to break the projections 218.

In some embodiments, the cutting of the projections 218 can be performed in a manner in which the distance (i.e., a vertical or axial distance in the direction of initial separation) between the substrate 212 and the template 201 can be further increased so as to break the protrusions.

In some embodiments, the cutting of the projections 218 can be performed in a manner in which a single edged razor blade or a thin, taut string (not shown in the figures) can be passed between the substrate 212 and the template 201 to break the projections 218.

However, care should be taken to ensure that the cutting method provides the desired degree of accuracy. Alternatively stated, the performance of some methods may not be able to provide a sufficient uniformity of the microneedles 220 after breaking the projections because the cutting of individual projections may not be sufficiently controlled.

Therefore, in accordance with at least some embodiments disclosed herein is the realization that a highly precise cutting method may be desirable for certain applications or indications. In some embodiments, as illustrated in FIGS. 19 and 20, the projections 218 can be cut using a focused laser 302. Thus, the length (i.e., height) of the microneedles 220 formed can be controlled accurately across the entire array of microneedles. Any suitable laser that can cut the solidified viscous polymer used for forming the projections 218 can be used for this purpose. For example, a continuous wave CO₂ laser 302 with a wavelength of 9.4 μm and 10.6 μm (i.e., in the infrared spectrum) may be used for polymer materials such as PVA.

In accordance with some embodiments, the method can be performed by adjusting a power of the laser 302 to a suitable or desired level based on the materials of the microstructure(s). For example, because cutting using such a focused infrared laser 302 is primarily a thermal process, the factors that may be considered when adjusting the power of the laser 302 include, but are not limited to, composition of the viscous polymer, the speed/rate at which the projections 218 are cut (i.e., the amount of time an individual projection 218 is exposed to the laser), tolerance to charring or oxidation at the tip of the microneedle (e.g., depending on whether the microneedle is pre-loaded with a therapeutic agent), possible blunting of the tip of the microneedle, possible localized “flow” at the tip of the microneedle due to heat, etc. For example, in some embodiments, PVA needles are cut to length while still containing high water content (approximately 10%-40% wt), which may reduce the tendency to oxidize or char. However, PVA may also be cut after fully drying (approximately <5% wt water) without charring at reduced power.

Some materials used for forming the microneedles 220, e.g., HA, may not be suitable for thermal cutting such as using an infrared laser, as they may readily oxidize and char if treated with such a laser. For such materials, an ultrafast pulsed laser may be used for cutting the projections.

For example, without wishing to be bound by theory, a laser pulse of 1 ns duration or shorter can produce a light intensity sufficient to produce a gaseous plasma as it interacts with the HA polymer surface, instantaneously evaporating the material in a so-called “ablative energy regime” as distinct from a “thermal energy regime.” In the ablative energy regime, material can be removed more quickly than thermal transfer processes can occur, leaving the surface relatively cool. The pulsed nature of the laser processing enables a long cooling so-called “dark” cycle, during which any thermal energy generated can dissipate in the bulk material. The combination of ultra-short ablative pulses and proportionally long dark periods makes laser processing of materials like HA very favorable in terms of preventing oxidation or charring during processing.

In accordance with some embodiments disclosed herein, the pulsed laser cut of HA as described herein produces needles that have a smooth surface and a sharp transition from non-cut to cut surface, i.e., squared-off, minimal radius at the cut origin. This may be advantageous in relation to skin penetration, presenting a microscopically sharp edge.

While materials like PVA are more tolerant of heat, with less susceptibility to oxidation than materials like HA, protrusions formed of materials like PVA can also be cut effectively with a pulsed laser system. In some embodiments, the ultrafast pulsed laser system includes a nanosecond pulsed ultraviolet laser (355 nm). Other examples include, but are not limited to, lasers having femtosecond pulse length range.

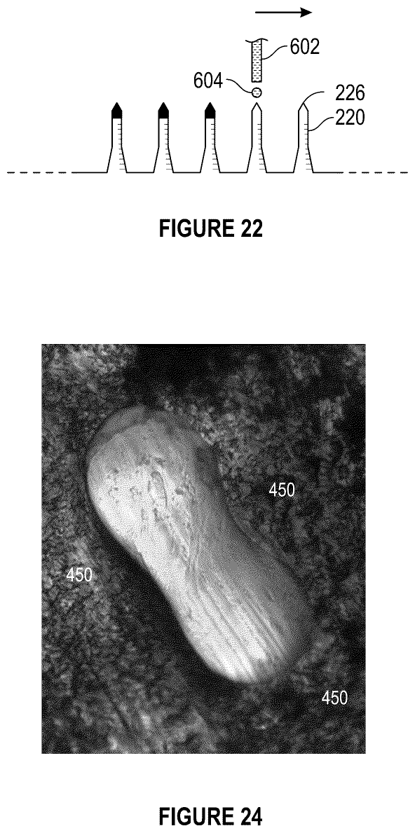

Referring now to FIG. 22, following the formation of microneedles 220, the now solidified viscous polymer layer 216 can be cut into individual dies which then form patches 224 to be applied to the subject. This can be accomplished by applying dicing cuts to divide the now solidified viscous polymer layer 216 disposed on the substrate 212 from which the microneedles 220 are pulled.

For example, in some embodiments, these cuts can be provided mechanically with a blade or blade-like cutter or using a dicing laser 304. The discussion relating to the applicability of various types of lasers for cutting polymer projections 218 to form the microneedles 220 can be applicable for the dicing cuts as well and will not be repeated herein for brevity, but is incorporated by a specific reference thereto.

Thus, in some embodiments, dicing cuts can be performed by a continuous infrared laser while in some embodiments the dicing cuts can be performed by an ultrafast laser, examples of both of which are discussed herein. Among the various considerations when selecting an appropriate laser for the dicing cuts is the material of the substrate 212, as will be appreciated by a person of skill in the art.

For example, in embodiments where a steel substrate is used, the steel substrate is normally unaffected by incident infrared laser light at this power, experiencing at most brief local heating and cooling. Thus, the substrate can be simply cleaned and reused for another cycle of microneedle manufacture after patches are removed. However, the nanosecond pulsed laser cuts produce such high peak energy that an underlying steel substrate 212 can be damaged during cutting. Thus, in instances where a nanosecond pulsed laser is more desirable, e.g., because of the material of the microneedles 220, a substrate 212 that is transparent to the wavelength of the pulsed laser such as, for example, quartz, may be used as the substrate 212.

In some embodiments, the microneedles 220 fabricated using the methods of the present disclosure are fabricated while still adherent to the underlying substrate 212. Individual patches 224, in various embodiments, can be cut (diced) from the continuous polymer layer 216 either while the layer is adherent to the substrate 212 or after it is removed. At some point, however, the layer must be removed from the substrate 212 for packaging. In some embodiments, this can be accomplished by use of a thin or sharpened edge, such as the sharp edge of a razor blade that is inserted under one edge of the polymer layer 216 at the contact point with the substrate 212.

Alternatively, an adhesive contact to an upper surface of the polymer layer 216 can be used to lift one edge of the layer 216. Without wishing to be bound by theory, it is contemplated that the polymer layer 216 can be strongly adherent to the substrate 212 if removed strictly normal to the surface of the substrate 212. However, if the polymer layer 216 is peeled away in a non-coplanar orientation relative to the substrate 212, it may separate relatively cleanly at the point of flexion between the lifted edge and the adherent, as yet planar area still attached to the substrate 212.

When fully dried, the polymer layer 216 can separate from a correctly finished substrate 212 without leaving behind any visible residue on the substrate 212. In some embodiments, the polymer layer 216 retains the micro-topology of the substrate surface 212, producing a slight opacity due to light scatter associated with the surface finish. However, in some embodiments, if a sacrificial separation layer (e.g., a release layer 214) is used, when the top-most needle-forming layer 216 is separated from the release layer 214, the needle-forming layer 216 can be observed to be relatively optically clear, not having been in direct contact with the textured substrate surface 212.

Incorporation of Drugs or Therapeutic Agents into the Microstructures

As disclosed herein, the composition used for the microstructure can comprise and/or be coated with one or more drugs or therapeutic agents. Some embodiments of methods for loading a drug or therapeutic agent onto the microstructure will be discussed herein below.

In accordance with some embodiments disclosed herein is the realization that because of the geometry and material properties of the microneedles 220, spontaneous wetting of the microneedles 220, e.g., by dipping the tips of the microneedles 220 into a solution in which a requisite therapeutic agent is dispersed, may be risky due to forces caused by surface tension at the interface of the tips and the solution. However, in some embodiments, contact loading can be accomplished by contacting the tips of the microneedle 220 structures to the fibrous tip of a marker pen charged with an aqueous solution in which a requisite therapeutic agent is dispersed, or by inserting the tip of each microneedle into the small aperture of a micropipet tip loaded with the aqueous solution.

In some embodiments, as discussed in detail with reference to FIG. 23, a non-contact method of loading is to dispense nanoliter-scale droplets 604 of the solution of the therapeutic agent into free space above the tip 226 of each microneedle 220. In some embodiments, a commercially available dispensing tool (e.g., a dispensing robot) is adapted to generate droplets 604 of a fixed volume (depending on the rheological properties of the solution of the therapeutic agent) as the dispensing head 602 moves across the uniformly spaced microneedles 220, such that the droplets fall regularly onto the tips 226 of the microneedle 220 structures.

In some embodiments, a microporous sponge (not explicitly shown) loaded with a solution including the therapeutic agent can be contacted with the tips 226 of the microneedles 220. Without wishing to be bound by theory, when an aqueous loading solution is charged into the interstitial spaces of a sponge material or a marker pen consisting of a set of parallel microfibers, the surface tension of the solution can be greatly reduced, as if it were wetted in the internal space of a capillary. In this reduced free energy state, the risk of spontaneous catastrophic wetting of the array can be virtually eliminated, and the sponge or marker can be applied to the microneedle arrays as an effective loading method.

In some embodiments, patches 224 of microneedles 220 loaded with a therapeutic agent are further dried to reduce the water content therein to prevent compromising the stability of the therapeutic agent and improving the rigidity of the microneedle structures. In some embodiments, the patches 224 are dried to reduce the water content to be less than about 5%, e.g., less than about 1%. The drying, in various embodiments, can be performed by placing the patches 224 under vacuum for a certain amount of time. For example, in some embodiments, the patches 224 are dried by placing the patches 224 under vacuum of about 20 mm Hg for about 12 hours.

The dried patches can then be packaged for storage. In some embodiments, microneedle patches 224 loaded with a therapeutic agent can be packaged in a thermoformed blister package. In some embodiments, additional headspace, insulating layers, or other precautions may be included in the design of the blister packages to remove the loaded tips from the hot surface normally used to fuse the foil backing of the blister package to the overlying thermoform plastic pockets so as to prevent thermal degradation of a heat-sensitive therapeutic agent loaded onto the microneedles. In some embodiments, to increase the robustness and storage stability of the microneedle patch loaded with a therapeutic agent, a desiccant and/or oxygen scavenger can be included in the blister package containing the microneedle patch.

In various embodiments, microneedle patches 224 manufactured using the methods described herein have, for example, less than about 5% microneedles deviating from normal (i.e., a line perpendicular to the surface of the solidified viscous polymer). Further the microneedle arrays formed using PVA as the viscous polymer are tolerant of levels of gamma irradiation consistent with sterilization (e.g., 18-50 kGy) with no visually discernible changes. In some embodiments, the microneedle patches manufactured using the methods described herein included a toxin complex (obotuluminum A) having an average molecular weight in a range from about 207 kDa to about 900 kDa.

The formed microneedles, in some embodiments, are provided with a therapeutic agent such as, for example, a formulation including onabotulinumtoxin A. Referring again to FIG. 22, a therapeutic agent can be applied directly to the microneedles using a dispensing device.

For example, in some embodiments, a non-contact method of loading can comprise dispensing nanoliter-scale droplets of the solution of the therapeutic agent into free space above the tip of each needle. Such a technique can be considered to be “drip loading” or “bolus dripping.” In some embodiments, a commercially available dispensing tool (e.g., a dispensing robot) is adapted to generate, e.g., approximately 6 nL droplets as the dispensing head moves across the uniformly spaced microneedles, such that the droplets fall regularly onto the tips of the needle structures.

Referring still to FIG. 23, in some embodiments, a piezoelectric actuator may deliver a pressure pulse perpendicular to the axis of a polyamide dispense tube 602 (“pipe” or dispensing head). The resulting compression shock ejects a droplet or bolus 604 from the tip of the pipe 602, which almost instantaneously refills by capillary action from a reservoir vertically above the pipe. In some embodiments, the same disposable pipet tip initially utilized to aliquot the loading solution may be reused for each run as the pipe reservoir, so as to minimize the surface area that might adsorb toxin from the loading solution. The robotic dispenser, in some embodiments, can use microstepper motors to provide high positional repeatability such that the bolus 604 is repeatably dispensed on different arrays of microneedles 220. Without wishing to be bound by theory, if a bolus 604 is released in a near-vertical trajectory in free-air, a well-formed microneedle structure readily captures the bolus 604 delivered from above.

In some embodiments, droplets deposit onto the strongly hydrophilic surface of the microneedle tips 226 and may typically remain localized to the top 30% of the needle structure (e.g., approximately 250 μm); droplets do not flow or splash down the sides of the needles, nor splash into smaller droplets. Splash and nonlocalized deposition may be readily apparent when droplet formation and ejection dynamics are not correctly set up, or in the case of irregularly spaced or malformed needles. The dosing of the microneedles using such method may be performed while the patches 224 are still attached to the underlying substrate or have been separated from the substrate, although being attached to the substrate may provide ease of handling and control. Factors that determine reproducible formation of the bolus 604 include, but are not limited to solution viscosity and surface tension.

In some embodiments, the microneedle structures may be formed from water-soluble polymers. In such microneedles, it might be expected that superficial application of droplets of aqueous solutions used to load the tips of the microneedles would cause dissolution, swelling, or collapse of the structures, rendering them deformed, dulled, or otherwise unsuitable for use as injection devices. Surprisingly, however, it was found that the tips of the microneedles formed as described are capable of absorbing the moisture from the droplets (as evidenced by the lack of splashing or drip patterns) without apparent ill effect.

Following the application of the bolus 604, when the structures are re-dried, no loss of integrity or skin penetration capability was observed. For example, when a model solution of sodium fluorescein was loaded onto the microneedles 220 and dried, and the microneedles 220 were cut so as to observe the tip cross-section, a fluorescence image showed that the fluorescent compound was distributed upon the exterior of the needle surface. Surprisingly, it was observed that the fluorescent compound had diffused also into the bulk material of the needle tip. Without wishing to be bound by theory, the diffusion may have occurred due to partial hydration of that material carrying the drug to the interior.

FIG. 24 shows a photomicrograph of a cross-sectional top view, taken along a longitudinal axis, of a microneedle loaded with a hydroalcoholic solution of sodium fluorescein. The diffusion of the payload (sodium fluorescein) into the interior of the microneedle structure can be clearly seen. The characteristic orange-red color of the concentrated fluorescein material is evident at the exterior surface (demarcated by the region 450, which extends around the perimeter of the microneedle cross section), while the interior of the needle structure is the bright green of a lower concentration of the material. The microneedle in the image was loaded with the hydroalcoholic solution of sodium fluorescein by the dip-loading method described herein. Thereafter, the microneedle was partially dried and mechanically cut with a razor blade to generate a cross-section. The image was obtained under white light, rather than by a fluorescent microscope, by imaging top-down, looking at the cut surface. It must be noted that while the microneedle seen in FIG. 24 has a peanut-shaped cross-section, microneedles with other cross-sectional shapes are expected to behave similarly in terms of payload diffusion.

One persistent issue in available coated microneedle products has been that the coating layer carrying the drug payload is prone to prematurely separate from the underlying microneedle structure due to the forces applied to these surfaces during skin penetration, leaving the drug payload at the skin surface without delivery to deeper layers. The diffusion of the drug payload into the interior of the soluble microneedles disclosed herein appears to render the loading solution continuously distributed into the microneedle tip, so that the risk of payload loss during skin penetration is vastly reduced. Thus, surprisingly, the hydration of the polymer materials from which the microneedles during the drug loading process does not pose a weakness. Instead, it confers an advantage in regard to effective delivery of the drug payload, rendered integral to the polymer structures by that same property of polymer hydration. From observation of fluorescein payloads, in some embodiments, the distribution of drug may be inferred to be maximal at the needle surface and minimal at the center of the coated tip of the needle, distributed consistent with diffusion laws. Such diffusion pattern is an identifiable characteristic of the presently disclosed microneedles. As the microneedle tips embed in the skin and hydrate to form intracutaneous depots, this drug distribution may further confer advantages in regard to drug release profiles, e.g., during subsequent depot clearance.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A method of manufacturing a microstructure, comprising: disposing a viscous polymer onto a substrate to form a continuous, viscous film layer; contacting a template against a surface of the viscous film layer, the template having a plurality of contact points contacting the viscous film layer surface; while urging air toward the viscous film layer from the template, separating the template from the viscous film layer surface to draw the viscous polymer into a plurality of projections; and permitting the plurality of projections to solidify.

Clause 2. The method of Clause 1, wherein the substrate comprises one of steel, copper, glass, quartz, and polymethylmethacrylate (PMMA).

Clause 3. The method of any one of the preceding Clauses, wherein the substrate has a surface roughness of about N16 or smoother.

Clause 4. The method of any one of the preceding Clauses, wherein dispensing the viscous polymer comprises pouring a predetermined amount of the viscous polymer on the substrate and drawing a bar across the substrate while maintaining a predetermined gap between the bar and the substrate.

Clause 5. The method of any one of the preceding Clauses, wherein the template comprises a plurality of pins extending from a base layer, and wherein the tips of the plurality of pins forming the plurality of contact points.

Clause 6. The method of any one of the preceding Clauses, wherein the template comprises a plurality of bumps raised from a base layer, the plurality of bumps being formed integrally with the base layer, the plurality of bumps forming the plurality of contact points.

Clause 7. The method of any one of the preceding Clauses, wherein the template comprises a plurality of bumps comprising a second viscous polymer disposed thereon, the plurality of bumps forming the plurality of contact points, and wherein the contacting the template comprises contacting the second viscous polymer against the viscous film layer.

Clause 8. The method of Clause 7, wherein the plurality of bumps further comprise a therapeutic agent disposed thereon, and wherein contacting the template comprises contacting the therapeutic agent against the viscous film layer.

Clause 9. The method of any one of the preceding Clauses, wherein the separating comprises separating the template from the surface of the viscous polymer at a predetermined rate.

Clause 10. The method of any one of the preceding Clauses, further comprising an intermediate viscous polymer layer disposed on the substrate intermediate the viscous polymer and the substrate, the intermediate viscous polymer layer comprising a second viscous polymer different from the viscous polymer.

Clause 11. The method of Clause 10, wherein the intermediate layer comprises ethyl cellulose (EtC) and the viscous polymer comprises one of hyaluronic acid or a salt thereof (HA) and polyvinyl alcohol (PVA).

Clause 12. The method of Clause 10, wherein the intermediate layer comprises PVA and the viscous polymer comprises HA.

Clause 13. The method of any one of the preceding Clauses, wherein the template further comprises outlet apertures spaced alternately with the plurality of contact points, and wherein the urging air toward the viscous film layer is performed using the outlet apertures.

Clause 14. The method of Clause 13, wherein each of the outlet apertures is fluidly coupled with a respective ingress channel at a base of each of the plurality of contact points.

Clause 15. The method of Clauses 14, wherein the permitting the plurality of projections to solidify comprises providing an airflow to projections through the outlet apertures during and/or after the drawing of the viscous polymer into the plurality of projections.

Clause 16. The method of Clause 15, wherein the urging air toward the viscous film layer comprises urging heated air toward the viscous film layer.

Clause 17. The method of any one of the preceding Clauses, wherein the viscous polymer comprises one or more of a viscous material, a biodegradable or biocompatible material, a solvent, and a plasticizer.

Clause 18. The method of any one of the preceding Clauses, wherein the viscous polymer comprises one of polyvinyl alcohol, and hyaluronic acid or a salt thereof.

Clause 19. The method of any one of the preceding Clauses, further comprising separating the solidified projections from the template to form the microstructure.

Clause 20. The method of Clause 17, wherein the separating the solidified projections comprises cutting the solidified projections using a blade.

Clause 21. The method of Clause 17, wherein the separating the solidified projections comprises cutting the solidified projections using an infrared laser

Clause 22. The method of Clause 17, wherein the separating the solidified projections comprises cutting the solidified projections using an ultrafast pulsed laser.

Clause 23. The method of any one of Clauses 17-20, further comprising contacting the microstructure with a therapeutic agent.

Clause 24. The method of Clause 21, wherein the therapeutic agent comprises a toxin complex having an average molecular weight in a range from about 207 kDa to about 900 kDa.

Clause 25. The method of any one of Clauses 17-22, further comprising irradiating the microstructure with gamma radiation.

Clause 26. A microstructure comprising protrusions from a surface of a continuous layer of a solidified viscous polymer formed by a method comprising: disposing the viscous polymer on a substrate; contacting a surface of the viscous polymer with a template having a plurality of contact points; drawing the viscous polymer at points of contact between the surface of the viscous polymer and the plurality of contact points while urging air toward the viscous film layer from the template to form the protrusions; permitting the protrusions to solidify; and separating the solidified protrusions from the template to form the microstructure.

Clause 27. The microstructure of Clause 26, further comprising a therapeutic agent disposed at distal ends of the microstructure away from the surface of the solidified viscous polymer.

Clause 28. The microstructure of Clause 27, wherein the therapeutic agent comprises a toxin complex having an average molecular weight in a range from about 207 kDa to about 900 kDa.

Clause 29. The microstructure of any one of Clauses 26-28, wherein the microstructure comprises microneedles, wherein less than 5% microneedles deviating from a line normal to the surface of the viscous polymer.

Clause 30. The microstructure of Clause 26, wherein the separating the solidified projections comprises cutting the solidified projections using a blade.

Clause 31. The microstructure of Clause 26, wherein the separating the solidified projections comprises cutting the solidified projections using an infrared laser.

Clause 32. The microstructure of Clause 26, wherein the separating the solidified projections comprises cutting the solidified projections using an ultrafast pulsed laser.

Clause 33. The microstructure of any one of Clauses 26-32, wherein the substrate comprises an intermediate layer disposed thereon, the intermediate layer comprising a second viscous polymer different from the viscous polymer.

Clause 34. The microstructure of Clause 33, wherein the intermediate layer comprises ethyl cellulose (EtC) and the viscous polymer comprises one of hyaluronic acid or a salt thereof (HA) and polyvinyl alcohol (PVA).

Clause 35. The microstructure of Clause 33, wherein the intermediate layer comprises PVA and the viscous polymer comprises HA.

Clause 36. The microstructure of any one of Clauses 26-33, wherein the template further comprises outlet apertures spaced alternately with the plurality of contact points, and wherein the method of forming the microstructure comprises urging air through the outlet apertures toward the viscous polymer.

Clause 37. The microstructure of Clause 36, wherein each of the outlet apertures is fluidly coupled with a respective ingress channel at a base of each of the plurality of contact points.

Clause 38. The microstructure of Clause 36, wherein the permitting the plurality of projections to solidify comprises providing an airflow to projections through the outlet apertures after the viscous polymer is drawn into the plurality of projections.

Clause 39. The microstructure of Clause 37, wherein the urging air toward the viscous film layer comprises urging heated air toward the viscous film layer.

Clause 40. The microstructure of any one of Clauses 26-39, wherein the viscous polymer comprises one of polyvinyl alcohol, and hyaluronic acid or a salt thereof.

Clause 41. The microstructure of any one of Clauses 26-40, wherein the substrate has a surface roughness of about N16 or smoother.

Clause 42. The microstructure of any one of Clauses 26-41, wherein the method further comprises cutting the solidified viscous polymer layer on which the microstructure is formed to form microstructure patches.

Clause 43. The microstructure of Clause 42, wherein the cutting the solidified viscous polymer layer comprises cutting the solidified viscous polymer layer using a blade.

Clause 44. The microstructure of Clause 42, wherein the cutting the solidified viscous polymer layer comprises cutting the solidified viscous polymer layer using a continuous infrared laser.

Clause 45. An apparatus for manufacturing a microstructure, comprising: a substrate carrier configured to carry a substrate; a template holder configured to carry a template having a plurality of contact points and enable a flow of air through outlet apertures disposed in the template; and an assembly configured to: enable the plurality of contact points to contact a surface of a viscous polymer layer disposed on the substrate provided on the substrate carrier; draw the viscous polymer at points of contact between the surface of the viscous polymer and the plurality of contact points to form protrusions of the viscous polymer; and permit the protrusions to solidify.

Clause 46. The apparatus of Clause 45, wherein the substrate carrier comprises a magnetic chuck configured to immobilize the substrate.

Clause 47. The apparatus of Clause 45, wherein the substrate carrier comprises a vacuum chuck configured to immobilize the substrate.

Clause 48. The apparatus of any one of Clauses 45-47, wherein the template holder comprises a vacuum chuck configured to immobilize the template.

Clause 49. The apparatus of any one of Clauses 45-47, wherein the template holder comprises a magnetic chuck configured to immobilize the template.

Clause 50. The apparatus of any one of Clauses 45-49, wherein the template comprises outlet apertures spaced alternately with the plurality of contact points.

Clause 51. The apparatus of Clause 50, wherein the template holder comprises an airflow ingress channel configured to provide air through the outlet apertures of the template.

Clause 52. The apparatus of any one of Clauses 45-51, wherein the assembly comprises a mechanism to move the template holder relative to the substrate carrier along a line perpendicular to a surface of the viscous polymer layer disposed on the substrate.

Clause 53. The apparatus of any one of Clauses 45-51, wherein the assembly comprises a mechanism to move the substrate carrier relative to the template holder along a line perpendicular to a surface of the viscous polymer layer disposed on the substrate.

Clause 54. The apparatus of any one of Clauses 52-53, wherein the mechanism comprises one or more of a motorized actuator, a pneumatic actuator and a piezoelectric actuator.

Clause 55. The apparatus of any one of Clauses 45-54, further comprising a laser configured to cut the solidified protrusions.

Clause 56. The apparatus of Clause 55, wherein the laser is a continuous infrared laser.

Clause 57. The apparatus of Clause 55, wherein the laser is an ultrafast pulsed laser.

Clause 58. The apparatus of any one of Clauses 45-57, further comprising a laser configured to cut the solidified viscous polymer layer on the substrate.

Clause 59. A method of manufacturing a microstructure, comprising: disposing a first water-soluble viscous polymer onto a substrate to form a first layer; disposing a second water-soluble viscous polymer onto the first layer to form a second layer; contacting a template against a surface of the second layer, the template having a plurality of contact points contacting the second layer surface; separating the template from the second layer surface to draw the second viscous polymer into a plurality of projections; and permitting the plurality of projections to solidify.

Clause 60. The method of Clause 59, wherein the first viscous polymer is different from the second viscous polymer.

Clause 61. The method of any one of Clauses 59-60, wherein the first viscous polymer comprises polyvinyl alcohol and the second viscous polymer comprises hyaluronic acid or a salt thereof.

Clause 62. The method of any one of Clauses 59-61, wherein the disposing comprises evenly spreading the second water-soluble viscous polymer over the first water-soluble viscous polymer.

Clause 63. The method of any one of Clauses 59-62, wherein when dried, the second layer does not spontaneously peel off of the first layer.

Clause 64. The method of any one of Clauses 59-63, wherein the first layer and the second layer are smooth, planar layers.

Clause 65. The method of any one of Clauses 59-64, further comprising peeling the microstructure from the substrate as a dual layer microstructure.

Clause 66. The method of any one of Clauses 59-65, wherein the first and second layers remain attached to the substrate without spontaneous peeling even when dried in a heated vacuum oven for 24 hours at −18 Hg vacuum at 40° C.

Clause 67. The method of any one of Clauses 59-66, wherein the first and second layers remain as manually separable separate layers.

Clause 68. The method of any one of Clauses 68-67, wherein the disposing the second water-soluble viscous polymer comprises overlapping the second layer onto the first layer with at least a portion of the second layer extending beyond a perimeter edge of the first layer to contact the substrate.

Clause 69. The method of Clause 68, wherein during drying, the second layer in contact with the substrate spontaneously peels away from the substrate.

Clause 70. The method of Clause 69, wherein the second layer delaminates from the substrate and causes the first layer to delaminate from the substrate.

Clause 71. A method of manufacturing a microstructure, comprising: disposing a viscous polymer onto the intermediate layer to form a continuous layer; contacting a template against a surface of the continuous layer, the template having a plurality of contact points contacting the continuous layer surface; separating the template from the continuous layer surface to draw the viscous polymer into a plurality of projections; permitting the plurality of projections to solidify; separating the plurality of contact points from the solidified projections to form a plurality of microneedles; and disposing a bolus of a therapeutic agent on a tip of each of the plurality of microneedles.

Clause 72. The method of Clause 71, wherein the disposing comprises contacting the bolus of the therapeutic agent directly with the tip.

Clause 73. The method of any one of Clauses 71-72, wherein the therapeutic agent comprises a toxin complex having an average molecular weight in a range from about 207 kDa to about 900 kDa.

Clause 74. The method of any one of Clauses 71-73, wherein separating the template from the continuous layer surface is performed while urging air toward the continuous film layer from the template.

### FURTHER CONSIDERATIONS

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The singular forms “a,” “an,” and “the” include plural referents unless the context clearly dictates otherwise. Thus, for example, reference to “a microneedle” includes reference to one or more microneedles, and reference to “the polymer” includes reference to one or more polymers.

In one or more aspects, the terms “about,” “substantially,” and “approximately” may provide an industry-accepted tolerance for their corresponding terms and/or relativity between items, such as from less than one percent to five percent.

The term “subject” refers to a mammal that may benefit from the administration using a transdermal device or method of this disclosure. Examples of subjects include humans, and other animals such as horses, pigs, cattle, dogs, cats, rabbits, and aquatic mammals.

As used herein, the term “active agent” or “drug” are used interchangeably and refer to a pharmacologically active substance or composition. Active agents in various embodiments may include small molecule drugs (e.g., nicotine), proteins (e.g., antigens, biologics, etc.), toxins (e.g., neurotoxins such as onabotulinumtoxin A), nucleic acids (e.g., siRNA, genetic vectors, etc.), diagnostic molecules (e.g., radioisotopes, superparamagnetic nanoparticles, etc.), allergens (e.g., extracts of pollen, nuts, egg, wheat, etc.), or combinations thereof.

The term “transdermal” refers to the route of administration that facilitates transfer of a drug into and/or through a skin surface wherein a transdermal composition is administered to the skin surface.

As used herein, the term “substantially” refers to the complete or nearly complete extent or degree of an action, characteristic, property, state, structure, item, or result.

As used herein, sequences, compounds, formulations, delivery mechanisms, or other items may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “therapeutic agent” means an agent utilized to treat, combat, ameliorate, prevent or improve an unwanted condition or disease of a patient. Additives such as permeation enhancers, controlled-release membranes, humectants, emollients, and the like may also be included in the therapeutic agent.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.5 to 10 g” should be interpreted to include not only the explicitly recited values of about 0.5 g to about 10.0 g, but also include individual values and sub-ranges within the indicated range. Thus, included in this numerical range are individual values such as 2, 5, and 7, and sub-ranges such as from 2 to 8, 4 to 6, etc. This same principle applies to ranges reciting only one numerical value. Furthermore, such an interpretation should apply regardless of the breadth of the range or the characteristics being described.

Unless otherwise defined, all technical and scientific terms used herein have the same meaning as commonly understood to one of ordinary skill in the art to which this disclosure belongs. Although any methods, devices and materials similar or equivalent to those described herein can be used in the practice or testing of the disclosure, representative methods, devices, and materials are described below.

The word “exemplary” is used herein to mean “serving as an example, instance, or illustration.” Any embodiment described herein as “exemplary” is not necessarily to be construed as preferred or advantageous over some embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes some embodiments not discussed in detail above. Various other modifications, changes and variations may be made in the arrangement, operation and details of the method and apparatus of the subject technology disclosed herein without departing from the scope of the present disclosure. Unless otherwise expressed, reference to an element in the singular is not intended to mean “one and only one” unless explicitly stated, but rather is meant to mean “one or more.” In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability. cm What is claimed is:

1. A method of manufacturing a microstructure, comprising:
disposing a viscous polymer onto a substrate to form a continuous, viscous film layer;
contacting a template against a surface of the viscous film layer, the template having a plurality of contact points contacting the viscous film layer surface;
while urging air toward the viscous film layer from the template, separating the template from the viscous film layer surface to draw the viscous polymer into a plurality of projections; and
permitting the plurality of projections to solidify thereby forming the microstructure.

2. The method of claim 1, wherein disposing the viscous polymer comprises pouring a predetermined amount of the viscous polymer on the substrate and drawing a bar across the substrate while maintaining a predetermined gap between the bar and the substrate.

3. The method of claim 1, wherein the template comprises a plurality of pins extending from a base layer, and wherein the tips of the plurality of pins form the plurality of contact points.

4. The method of claim 1, wherein the template comprises a plurality of bumps comprising a second viscous polymer disposed thereon, the plurality of bumps forming the plurality of contact points, and wherein the contacting the template comprises contacting the second viscous polymer against the viscous film layer.

5. The method of claim 1, further comprising an intermediate viscous polymer layer disposed on the substrate intermediate the viscous polymer and the substrate, the intermediate viscous polymer layer comprising a second viscous polymer different from the viscous polymer.

6. The method of claim 1, wherein the viscous polymer comprises one or more of a viscous material, a biodegradable or biocompatible material, a solvent, and a plasticizer.

7. The method of claim 1, wherein the viscous polymer comprises one of polyvinyl alcohol, and hyaluronic acid or a salt thereof.

8. The method of claim 1, further comprising separating the solidified projections from the template to form the microstructure.

9. The method of claim 8, wherein the separating the solidified projections comprises cutting the solidified projections using a laser.

10. The method of claim 8, further comprising contacting the microstructure with a therapeutic agent.

11. A microstructure comprising protrusions from a surface of a continuous layer of a solidified viscous polymer formed by a method comprising:
disposing the viscous polymer on a substrate;
contacting a surface of the viscous polymer with a template having a plurality of contact points;
drawing the viscous polymer at points of contact between the surface of the viscous polymer and the plurality of contact points while urging air toward the viscous film layer from the template to form the protrusions;
permitting the protrusions to solidify; and
separating the solidified protrusions from the template to form the microstructure.

12. The microstructure of claim 11, further comprising a therapeutic agent disposed at distal ends of the microstructure away from the surface of the solidified viscous polymer.

13. The microstructure of claim 11, wherein the substrate comprises an intermediate layer disposed thereon, the intermediate layer comprising a second viscous polymer different from the viscous polymer.

14. The microstructure of claim 13, wherein the intermediate layer comprises PVA and the viscous polymer comprises HA.

15. The microstructure of claim 11, wherein the template further comprises outlet apertures spaced alternately with the plurality of contact points, and wherein the method of forming the microstructure comprises urging air through the outlet apertures toward the viscous polymer.

16. The microstructure of claim 11, wherein the viscous polymer comprises one of polyvinyl alcohol, and hyaluronic acid or a salt thereof.

17. An apparatus for manufacturing a microstructure, comprising:
a substrate carrier configured to carry a substrate;
a template holder configured to carry a template having a plurality of contact points and enable a flow of air through outlet apertures disposed in the template; and
an assembly configured to:
enable the plurality of contact points to contact a surface of a viscous polymer layer disposed on the substrate provided on the substrate carrier;
draw the viscous polymer at points of contact between the surface of the viscous polymer and the plurality of contact points to form protrusions of the viscous polymer; and
permit the protrusions to solidify.

18. The apparatus of claim 17, wherein the template comprises outlet apertures spaced alternately with the plurality of contact points.

19. The apparatus of claim 18, wherein the template holder comprises an airflow ingress channel configured to provide air through the outlet apertures of the template.

20. The apparatus of claim 17, further comprising a laser configured to cut the solidified protrusions.
